(19) United States
(12) Patent Application Publication
(10) Pub. No.: US 2022/0242874 A1
(43) Pub. Date: Aug. 4, 2022

(54) CAMPTOTHECIN DERIVATIVES

(71) Applicant: SUN PHARMA ADVANCED RESEARCH COMPANY LIMITED, Mumbai, Maharashtra (IN)

(72) Inventors: Jiten Ranchhodbhai PATEL, Vadodara, Gujarat (IN); Gopalkumar Chimanlal PATEL, Vadodara, Gujarat (IN); Omkar Prakash GORE, Maharashtra (IN); Prabal SENGUPTA, Vadodara, Gujarat (IN); Trinadha Rao CHITTURI, Vadodara, Gujarat (IN)

(73) Assignee: SUN PHARMA ADVANCED RESEARCH COMPANY LIMITED, Mumbai, Maharashtra (IN)

(21) Appl. No.: 17/625,960

(22) PCT Filed: Jul. 13, 2020
(86) PCT No.: PCT/IB2020/056580

§ 371 Date: Jan. 10, 2022

(30) Foreign Application Priority Data
Jul. 11, 2019 (IN) 201921027783

(51) Int. Cl. C07D 491/22; A61P 35/00

(52) U.S. Cl. CPC C07D 491/22; A61P 35/00

(57) ABSTRACT

A compound of Formula I or a pharmaceutically acceptable salt thereof (wherein X, Y, Z and n are defined herein). These compounds are useful in the treatment of diseases mediated by topoisomerase I enzyme such as cancers. Also provided are processes for the preparation of compounds of Formula I. The compounds are more water soluble, stable in buffer solution at various pH, and exhibit better anti-tumor activity and rapid release of SN-38 in tumor microenvironments.

### RELATED APPLICATIONS

This application claims priority to Indian Provisional Patent Application No. 201921027783 filed on Jul. 11, 2019, which is hereby incorporated by reference.

### FIELD OF THE INVENTION

This invention provides a compound of Formula I:

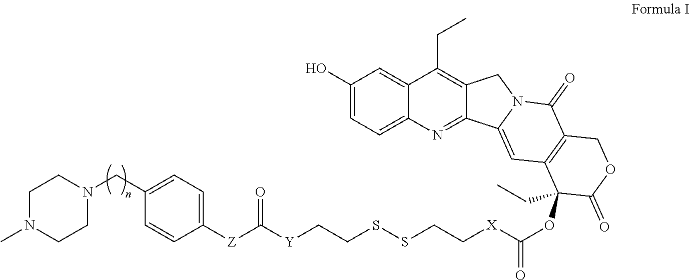

or a pharmaceutically acceptable salt thereof (wherein X, Y, Z and n are as defined herein). These compounds are useful in the treatment of diseases mediated by topoisomerase I enzyme such as cancers. The present invention also provides processes for the preparation of compounds of Formula I.

### BACKGROUND OF THE INVENTION

Camptothecin, a plant alkaloid isolated from Camptotheca acuminata (family Nyssaceae), was first discovered in the early 1960s. Camptothecin and its derivatives are potent topoisomerase I inhibitors with strong antitumor activities both in vitro and in vivo. It was discovered that the lactone ring of camptothecin is beneficial for specific interaction with topoisomerase I and selective antitumor activity. Because of severe and unpredictable side effects of camptothecin in early clinical studies, clinical development was halted in the 1970s. It was later revealed that the water insolubility of camptothecin was an important factor mediating the unpredictable toxic effects (Clin. Cancer Res., 2001, 7, 2182-2194). Several derivatives of camptothecin with improved solubility have been synthesized, including irinotecan, topotecan and belotecan.

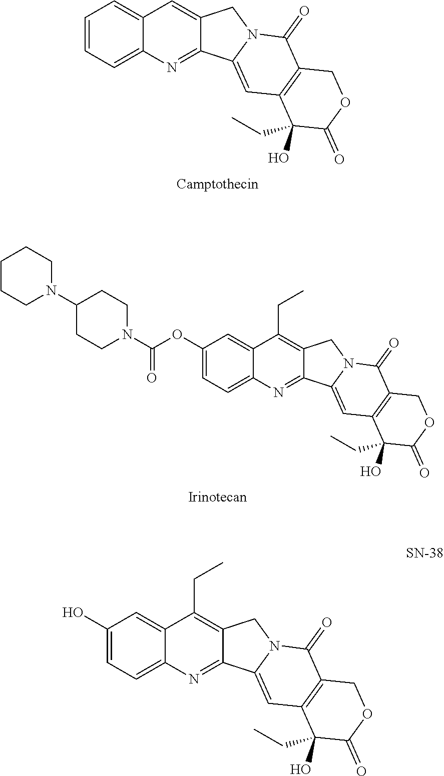 [SMILES: CC[C@@]1(O)C(=O)OCc2c1cc1n(c2=O)Cc2cc3ccccc3nc2-1 ; CCc1c2c(nc3ccc(OC(=O)N4CCC(N5CCCCC5)CC4)cc13)-c1cc3c(c(=O)n1C2)COC(=O)[C@]3(O)CC ; CCc1c2c(nc3ccc(O)cc13)-c1cc3c(c(=O)n1C2)COC(=O)[C@]3(O)CC]

Irinotecan was approved in the U.S. in 1996 (as irinotecan hydrochloride), marketed under the tradename Camptosar®, indicated for the treatment of metastatic carcinoma of the colon or rectum. However, only about 2-8% of the pro-drug is converted to SN-38 (the active metabolite of irinotecan) by carboxylesterases present in liver and cancer cells. Accordingly, a high dose of irinotecan needs to be administered to achieve the desired therapeutic effect. For example, Camptosar® has to be injected at a dose of 125-180 mg/m² intravenously over a period of 90 minutes to treat colorectal cancer. The conversion of irinotecan to SN-38 is highly variable among patients. It is believed that the low bioconversion efficiency from irinotecan to the active form SN-38 is responsible for high interpatient variability in terms of the pharmacokinetics, which leads to considerable individual variation in efficacy and toxicity. The clinical application of irinotecan is also limited by its toxic, dose-related side effects, such as early or late forms of diarrhea, neutropenia, myelosuppression, and pulmonary toxicity.

SN-38 is an approximately 1000 times more potent metabolite of irinotecan. About 96% of SN-38 is protein bound in plasma (See Camptosar® Prescribing Information approved by USFDA). However, the clinical use of the SN-38 is limited by its poor aqueous solubility and conversion of the pharmacologically active lactone ring into an inactive carboxylate form at pH greater than 6. Thus, inherent poor water solubility and stability has led others to develop new derivatives of SN-38 which overcomes these drawbacks. For example, EZN2208, which was in a Phase II trial for metastatic breast cancer, has a four-arm polyethylene glycol (PEG) conjugation at the C₂₀ position of SN-38 to increase water-solubility. However, the most common reported drug-related adverse events were diarrhea, nausea and neutropenia. Another clinical candidate NK-012 (in Phase II study), has hydrophilic PEG bound via a hydrophobic polyglutamate linker at the C-10 position of SN-38. It self-assembles into micelles in aqueous solution.

Various pro-drugs of camptothecin and/or SN-38 and its derivatives are disclosed in, for example, U.S. Pat. Nos. 7,452,900, 9,150,585, 10,098,967, 7,875,602, 9,206,192, 9,266,911, 9,480,756 and 6,350,756, International Publication Nos. WO 2018/171164, WO 2003/043584, WO 2015/178265A1, WO 120/67670A1 and WO 2016/045505A1; Chinese Publication Nos. CN 103508981A, CN 104368011A, CN 105131039A, CN 104370862A, CN 108785683A, CN 108586535A, CN 1035520110A, CN 103524519A, CN 105457038A, CN 106046029A, CN 106916236A, CN 106620717A, CN 106967081A and CN 108409756A, and Korean Publication No. KR 2014010517.

There is a clear and continuing need for novel derivatives of camptothecin that exhibit improved solubility and stability and reduced toxicity while retain the desired pharmacological activity.

### SUMMARY OF THE INVENTION

In one aspect, the present invention relates to a compound of Formula I

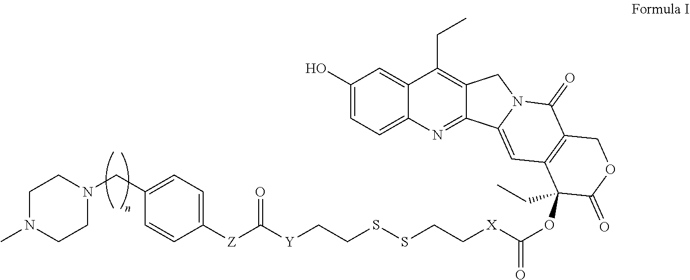

or a pharmaceutically acceptable salt thereof, wherein

### X is —NH—, —O— or —CH₂—;

### Y is —NH—, —O— or —CH₂—;

Z is absent, —NH— or —N(C₁₋₃ alkyl)-; and
n is an integer selected from 0 or 1.

The compounds of the present invention have good water solubility and are stable in buffer solution at various pH (for e.g. at pH ranging from 4.7 to 7.4). The compounds of Formula I exhibit potent inhibition of cell growth in NCI H69, NCI H187, NCI H526, PANC-1, MDA-MB-231 cells, MX-1 cells and MDA-MB468 cell lines demonstrating their utility in the treatment of cancer.

### DETAILED DESCRIPTION OF THE INVENTION

### Definitions

“Pharmaceutically acceptable salt” as used herein includes acid addition salts formed with either organic or inorganic acids. Suitable pharmaceutically acceptable salts of the compounds of the invention include, but are not limited to, acid addition salts which may be salts of inorganic acids such as hydrochloric acid, hydrobromic acid, and phosphoric acid, or of organic acids such as, for example, acetic acid, benzenesulfonic acid, methanesulfonic acid, benzoic acid, citric acid, glycolic acid, lactic acid, fumaric acid, succinic acid, adipic acid, pimelic acid, suberic acid, azelaic acid, malic acid, tartaric acid, and amino acids such as glutamic acid or aspartic acid. The pharmaceutically acceptable acid addition salt of the compounds of the present invention includes salts formed with the addition of one or more equivalents of acid, for example, monohydrochloride, and dihydrochloride salts.

The term “alkyl” as used herein refers to a saturated hydrocarbon chain radical that includes solely carbon and hydrogen atoms in the backbone, either linear or branched and which is attached to the rest of the molecule by a single bond, e.g., methyl, ethyl, n-propyl, and 1-methylethyl (isopropyl). The alkyl chain may have 1 to 3 carbon atoms unless specified otherwise.

The numerical in phrases like “C₁₋₃”, refers to 1 to 3 carbon atoms in the chain. For example, the phrase “C₁₋₃ alkyl” refers to an alkyl chain having 1 to 3 carbon atoms.

The term “effective amount” as used herein refers to an amount of the compound which is sufficient, upon single or multiple dose administration(s) to a subject, in curing, alleviating, relieving or partially addressing the clinical manifestation of a given disease or state and its complications beyond that expected in the absence of such treatment. Thus, the result can be reduction and/or alleviation of the signs, symptoms, or causes of a disease, or any other desired alteration of a biological system. It is understood that “a therapeutically effective amount” can vary from subject to subject depending on age, weight, general condition of the subject, the condition being treated, the severity of the condition being treated, and the judgment of the prescribing physician.

The term “treating or treatment” as used herein refers to completely or partially curing, alleviating, ameliorating, improving, relieving, delaying onset of, inhibiting progression of, reducing severity of, and/or reducing incidence of one or more symptoms or features of a particular disease, disorder, and/or condition.

The term “subject” as used herein refer to either a human or a non-human animal. These terms include mammals such as humans, primates, livestock animals (e.g., bovines and porcines), companion animals (e.g., canines and felines) and rodents (e.g., mice and rats).

In one aspect, the present invention relates to a compound of Formula I

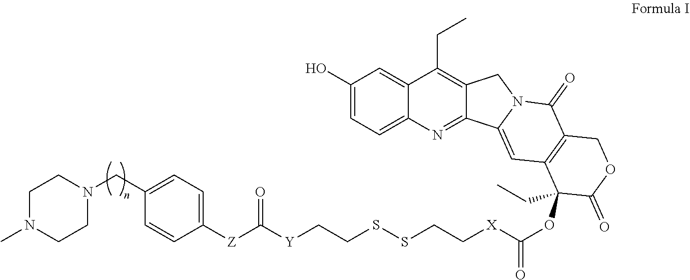

or a pharmaceutically acceptable salt thereof, wherein

### X is —NH—, —O— or —CH₂—;

### Y is —NH—, —O— or —CH₂—;

Z is absent, —NH— or —N(C₁₋₃ alkyl)-; and
n is an integer selected from 0 or 1.

The present invention may involve one or more embodiments. It is to be understood that the embodiments below are illustrative of the present invention and are not intended to limit the claims to the specific embodiments exemplified. It is also to be understood that the embodiments defined herein may be used independently or in conjunction with any definition, any other embodiment defined herein. Thus the invention contemplates all possible combinations and permutations of the various independently described embodiments.

According to one embodiment, the present invention provides a compound of Formula I, wherein X is —O—;

### Y is —NH— or —O—;

Z is absent, —NH— or —N(C₁₋₃ alkyl)- and
n is an integer selected from 0 or 1.
In another embodiment, the present invention provides a compound of Formula I, wherein X is —O—; Y is —O—; Z is —NH— or —N(C₁₋₃ alkyl) and n is integer 0.

In another embodiment, the present invention provides a compound of Formula I, wherein X is —NH—. In another embodiment, X is —O—. In yet another embodiment, X is —CH₂—.

In another embodiment, the present invention provides a compound of Formula I, wherein Y is —NH—. In another embodiment, Y is —O—. In yet another embodiment, Y is —CH₂—.

In another embodiment, the present invention provides a compound of Formula I, wherein Z is absent. In yet another embodiment, Z is —NH—. In yet another embodiment, Z is —N(C₁₋₃ alkyl)-. In yet another embodiment, Z is —N(CH₃)—.

In yet another embodiment, the present invention provides a compound of Formula I, wherein n is 1. In yet another embodiment, n is 0.

In another embodiment, the present invention provides a compound of Formula I, wherein

### X is —O—;

### Y is —O—;

Z is —N(C₁₋₃ alkyl)-; and
n is 0.

In yet another embodiment, the present invention provides a compound of Formula I, wherein

### X is —O—;

### Y is —O—;

### Z is —N(CH₃)—; and

n is 0.

In another embodiment of the present invention, the compound of Formula I is selected from:

4-[3 -(4-(4-Methylpiperazin-1 -yl)phenylcarbamoyl)propyldisulfanyl][4S)-4,11-diethyl-3,4,12,14-tetrahydro-9-hydroxy-1H-pyrano[3′,4′:6,7]indolizino[1,2-b]quinoline-3,14-(4H,12H)dione-4-yl]butyrate;
2-(2-{N-[4-(4-Methylpiperazin-1-yl)phenyl]carbamoyloxy}ethyldisulfanyl)ethyl [(4S)-4,11-diethyl-3,4,12,14-tetrahydro-9-hydroxy-1H-pyrano[3′,4′:6,7] indolizino[1,2-b]quinoline-3,14-(4H,12H)dione-4-yl]carbonate;
2-(2-{N-Methyl-N[4-(4-(4-methylpiperazin-1-yl)phenyl]carbamoyloxy} ethyldisulfanyl)ethyl [4S)-4,11-diethyl-3,4,12,14-tetrahydro-9-hydroxy-1H-pyrano[3′,4′:6,7]indolizino[1,2-b]quinoline-3,14-(4H,12H)dione-4-yl]carbonate;
2-(2-{4-[4-Methylpiperazin-1-yl]benzoylamino}ethyldisulfanyl)ethyl [4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate;
2-(2-{4-[4-Methylpiperazin-1-ylmethyl]benzoylamino}ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate;
[2-(2-{3-[4(4-Methylpiperazin-1-yl)phenyl]ureido}ethyldisulfanyl)ethyl] (4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbamate;
and pharmaceutically acceptable salts thereof.

In another embodiment, the compound of Formula I is selected from:

4-[3-(4-(4-Methylpiperazin-1 -yl)phenylcarbamoyl)propyldisulfanyl][(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]butyrate hydrochloride;
2-(2-{N-[4-(4-Methylpiperazin-1-yl)phenyl]carbamoyloxy}ethyldisulfanyl)ethyl [(4S)-4,11-diethyl-3,4,12,14-tetrahydro-9-hydroxy-1H-pyrano[3′,4′:6,7] indolizino[1,2-b] quinoline-3,14-(4H,12H)dione-4-yl]carbonate hydrochloride;
2-(2-{N-Methyl-N-[4(4-methylpiperazin-1-yl)phenyl]carbamoyloxy} ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]carbonate hydrochloride;
2-(2-{N-Methyl-N-[4-(4-(4-methylpiperazin-1-yl)phenyl]carbamoyloxy} ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]carbonate dihydrochloride;
2-(2-{4-[4-Methylpiperazin-1-yl]benzoylamino}ethyldisulfanyl)ethyl [4S)-4,11 -diethyl-3,4,12,14-tetrahydro-9-hydroxy-1H-pyrano[3′,4′:6,7]indolizino[1,2-b]quinoline-3,14-(4H,12H)dione-4-yl] carbonate dihydrochloride;
2-(2-{4-[4-Methylpiperazin-1-ylmethyl]benzoylamino}ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate dihydrochloride; and
[2-(2-{3-[4-(4-Methylpiperazin-1-yl)phenyl]ureido}ethyldisulfanyl)ethyl] [(4S)-4,11-diethyl-3,4,12,14-tetrahydro-9-hydroxy-1H-pyrano[3′,4′:6,7] indolizino[1,2-b]quinoline-3,14-(4H,12H)dione-4-yl] carbamate hydrochloride.

In another aspect, the present invention relates to a compound of Formula Ia

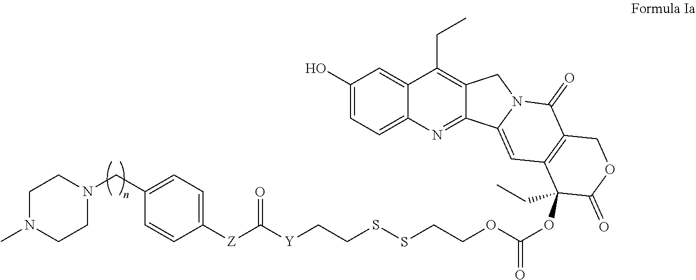

or a pharmaceutically acceptable salt thereof, wherein

### Y is —NH— or —O—;

Z is absent, —NH— or —N(C₁₋₃ alkyl)- and
n is an integer selected from 0 or 1.

Another embodiment is a compound of Formula Ia wherein Y is —NH—. Another embodiment is a compound of Formula Ia wherein Y is —O—.

Another embodiment is a compound of Formula Ia wherein Z is absent. Yet another embodiment is a compound of Formula Ia wherein Z is —NH—. Yet another embodiment is a compound of Formula Ia wherein Z is —N(C₁₋₃ alkyl)-. Another embodiment is a compound of Formula Ia wherein Z is —N(CH₃)—.

Yet another embodiment is a compound of Formula Ia wherein n is 1. Yet another embodiment is a compound of Formula Ia wherein n is 0.

In another embodiment, the present invention provides a compound of Formula Ia, wherein

### Y is —O—;

Z is —N(C₁₋₃ alkyl)-; and
n is 0.

In yet another embodiment, the present invention provides a compound of Formula Ia, wherein Y is —O—;

### Z is —N(CH₃)—; and

n is 0.

In another aspect, the present invention provides a compound of Formula Ib:

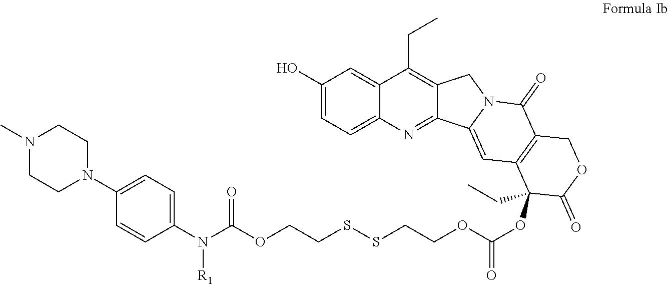

or a pharmaceutically acceptable salt thereof, wherein
R₁ is hydrogen or C₁₋₃ alkyl.

Another embodiment is a compound of Formula Ib wherein R₁ is hydrogen. Yet another embodiment is a compound of Formula Ib wherein R₁ is methyl.

The compounds described herein are topoisomerase I inhibitors and therefore are believed to be useful as medicaments, particularly for the treatment of diseases or disorders that benefit from the inhibition of topoisomerase I. In particular, the compounds described herein exhibit antiproliferative activity and are therefore used on account of their therapeutic activity and possess physicochemical properties that make them suitable for formulation in pharmaceutical compositions. The compounds of the present invention are expected to be useful in the treatment of a number of tumors and/or cancers including, but not limited to, lung cancer (including non-small-cell lung cancer and small-cell lung cancer), breast cancer (including triple-negative breast cancer and non-triple-negative breast cancer), colon cancer, rectal cancer, prostate cancer, melanoma, pancreatic cancer, stomach cancer, liver cancer, brain cancer, kidney cancer, cancer of the uterus, cancer of the cervix, ovarian cancer, cancer of the urinary tract, gastrointestinal cancer, urothelial cancer, head and neck cancer, thyroid cancer, esophageal cancer, endometrial cancer, and cholangiocarcinoma.

Thus, in another aspect, the present invention provides a method of treatment of diseases or disorders mediated by topoisomerase I enzyme by administering to a subject in need thereof an effective amount of a compound of Formula I, a compound of Formula Ia, a compound of Formula Ib, or a pharmaceutically acceptable salt thereof. In one embodiment, the subject is human.

In another embodiment, the present invention provides a method of treatment of a cell proliferative disease by administering to a subject in need thereof an effective amount of a compound of Formula I, a compound of Formula Ia, a compound of Formula Ib, or a pharmaceutically acceptable salt thereof. In another embodiment, the subject is a human.

In another embodiment, the present invention provides a method of treatment of a cancer selected from a group consisting of lung cancer (including non-small-cell lung cancer and small-cell lung cancer), breast cancer (including triple-negative breast cancer and non-triple-negative breast cancer), colon cancer, rectal cancer, prostate cancer, melanoma, pancreatic cancer, stomach cancer, liver cancer, brain cancer, kidney cancer, cancer of the uterus, cancer of the cervix, ovarian cancer, cancer of the urinary tract, gastrointestinal cancer, urothelial cancer, head and neck cancer, thyroid cancer, esophageal cancer, endometrial cancer, and cholangiocarcinoma, comprising administering to a subject in need thereof an effective amount of a compound of Formula I, compound of Formula Ia, compound of Formula Ib, or a pharmaceutically acceptable salt thereof. In another embodiment, the subject is a human.

In another embodiment, the present invention provides a method of treatment of a cancer selected from a group consisting of non-small cell lung cancer, colon cancer, rectal cancer, pancreatic cancer, breast cancer and prostate cancer, comprising administering to a subject in need thereof an effective amount of a compound of Formula I, a compound of Formula Ia, a compound of Formula Ib, or a pharmaceutically acceptable salt thereof. In another embodiment, the subject is a human.

In another embodiment, the present invention provides a method of treatment of a cancer selected from a group consisting of non-small cell lung cancer, triple negative breast cancer, ovarian cancer, colon cancer and cholangiocarcinoma, comprising administering to a subject in need thereof an effective amount of a compound of Formula I, compound of Formula Ia, compound of Formula Ib, or a pharmaceutically acceptable salt thereof. In another embodiment, the subject is a human.

The compounds of the invention may be formulated into a composition that additionally comprises suitable pharmaceutically acceptable carriers, including excipients and other compounds that facilitate administration of the compound to a subject. Such pharmaceutical compositions and processes for preparing the same are described, e.g., in Remington: The Science and 50 Practice of Pharmacy (D. B. Troy, Editor, 21st Edition, Lippincott, Williams & Wilkins, 2006). The compounds and compositions described herein may be administered orally, parenterally, intramuscularly, transdermally or intravenously.

Thus, in one embodiment, the present invention provides a pharmaceutical composition comprising a compound of Formula I, a compound of Formula Ia or a compound of Formula Ib, or a pharmaceutically acceptable salt thereof, with a pharmaceutically acceptable carrier, diluent, or excipient.

### Methods of Preparation

The compounds of Formula I, wherein X and Y are same or different and each independently represents —NH— or —O—; and Z is absent, —NH— or —N(C₁₋₃alkyl)-, can be synthesized by condensation of a compound of Formula IIa, wherein L is a leaving group (such as halide, phenoxy, 4-nitrophenoxy, chloroethoxy, 1-imidazolyl) and P is protecting group, such as tert-butyloxycarbonyl, tert-butyldimethylsilyl, tert-butyldiphenylsilyl, or methoxymethyl acetal, with a compound of Formula III, wherein X and Y are independently selected from —NH— or —O—; Z is absent, —NH— or —N(C₁₋₃ alkyl)- and n is an integer selected from 0 or 1, in the presence of a base, optionally in conjunction with a suitable catalyst (such as, e.g., 4-(N,N-dimethylamino)pyridine or 1-hydroxybenzotriazole) in a suitable solvent to provide a compound of Formula IV (wherein X and Y are independently selected from —NH— or —O—, Z is absent, —NH— or —N(C₁₋₃ alkyl)- and n is 0 or 1), which then can be deprotected to yield a compound of Formula I. Compounds of Formulas Ia and Ib can be prepared by a similar method as described above.

The process can be depicted as shown in Scheme-1 below.

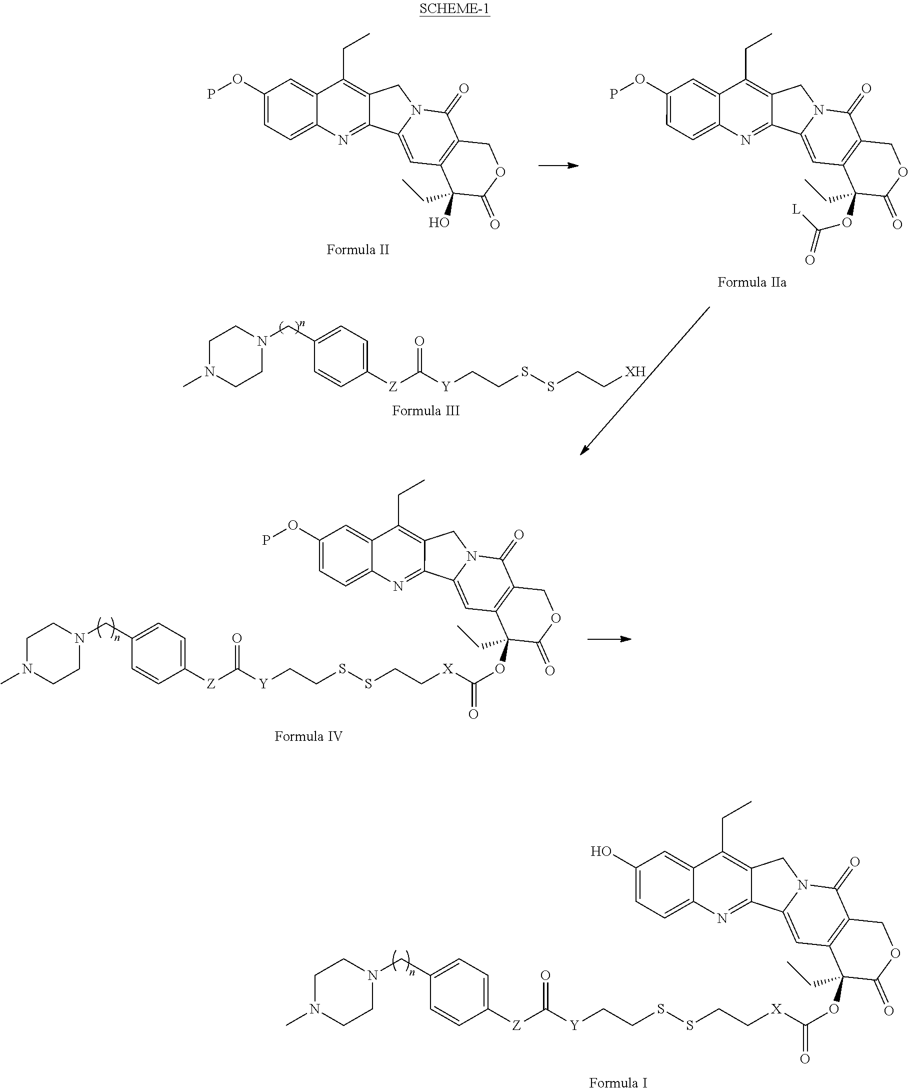

Compounds of Formula IIa can be synthesized from the compound of Formula II, wherein P is as defined above, by using any carbonylating reagent, such as phenyl chloroformate, 4-nitrophenyl chloroformate, Phosgene, diphosgenes, trifluoroethyl chloroformate or carbonyldiimidazole, commonly known for such purpose. Optionally, the compound of Formula IIa may be prepared in situ without prior isolation and reacted with the compound of Formula III. The general methods for this purpose are well known to those skilled in the art. Some of the commonly used methods include treatment of the compound of Formula II with the following reagents:
Phosgene, diphosgenes, or triphosgenes to obtain a compound of Formula Ha, wherein L is Cl.
An aryl chloroformate such as phenyl chloroformate or 4-nitrophenyl chloroformate to obtain a compound of Formula IIa, wherein L is phenoxy or 4-nitrophenoxy.
A haloalkyl chloroformate, such as trifluoroethyl chloroformate or chloroethyl chloroformate to obtain a compound of Formula IIa, wherein L is trifluoroethoxy or chloroethoxy.
A carbonyl diheterocyclyl compound such as carbonyldiimidazole to obtain a compound of Formula IIa, wherein L is 1-imidazolyl.
A N-hydroxyheterocyclyl choroformate such as N-hydroxysuccinimidyl chloroformate to obtain a compound of Formula IIa, wherein L is N-hydroxysuccinimidyl.

The carbonylation reaction may be performed in the presence or absence of an inert base, optionally in conjunction with a suitable catalyst in a suitable solvent such as methylene dichloride, toluene or tetrahydrofuran.

Compounds of Formula III wherein X and Y are independently selected from —NH— or —O—; Z is —NH— or —N(C₁₋₃ alkyl)- and n is an integer selected from 0 or 1, can be synthesized from compounds of Formula IIIa, wherein Z is —NH— or —N(C₁₋₃ alkyl)- and n is an integer selected from 0 or 1 by using any carbonylating reagent commonly known for such purpose, for example as described above, to provide a compound of Formula IIIa′, wherein L is a leaving group, which is then reacted with a compound of Formula V, wherein X and Y are independently selected from —NH— or —O—, in a suitable solvent to yield the compound of Formula III. The process can be depicted as shown in Scheme-1A below.

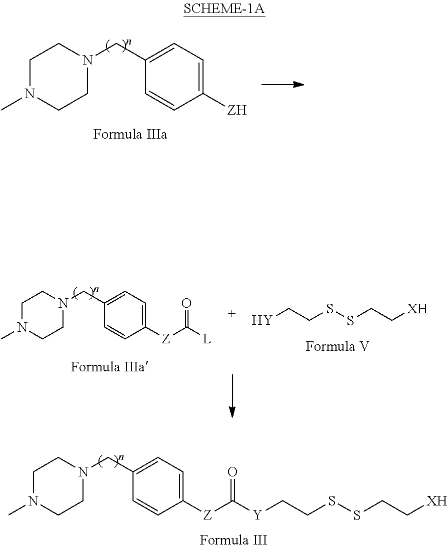

Optionally, the compound of Formula IIIa′ may be prepared in situ without prior isolation and reacted with a compound of Formula V.

Compounds of Formula III, wherein X and Y are independently selected from —NH— or —O—; Z is absent and n is an integer selected from 0 or 1, can be synthesized by condensation of a compound of Formula IIIb, wherein n is an integer selected from 0 or 1, with a compound of Formula V, wherein X and Y are independently selected from —NH— or —O—, in a suitable solvent to yield the compound of Formula III. The process can be depicted as shown in Scheme-1B below.

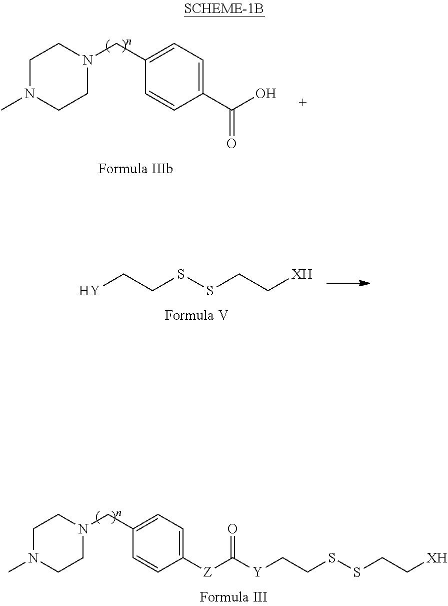

The condensation reaction can be carried out in a manner known in art, the reaction conditions being dependent on how the acid group of Formula IIIb has been activated, usually in the presence of a suitable aprotic solvent or diluent or of a mixture thereof and, if necessary, in the presence of a condensation agent and in the presence or absence of a base. Customary condensation agents include, for example, carbodiimides such as NN′-diethyl-, N,N′-diisopropyl, N,N′-dicyclohexyl- or N-ethyl-N′-(3-diethylaminopropyl)carbodiimide, suitable carbonyl compounds, for example carbonyldiimidazole, suitable 1,2-oxazolium compounds, for example 2-ethyl-5-phenyl-1,2-oxazolium 3′-sulfonate and 2-tert-butyl-5-methyl-isoxazolium perchlorate, or a suitable acylamino compound, for example, 2-ethoxy-1-ethoxycarbonyl-1,2-dihydroquinoline. The bases normally used for aiding the condensation are either inorganic bases such as sodium or potassium carbonate, or organic bases, such as pyridine, triethylamine, N,N-diisopropylethylamine or 4-(dimethylamino)pyridine.

Alternatively, the compound of Formula I, wherein X and Y are same or different and each independently represents —NH— or —O— and Z is —NH— or —N(C₁₋₃ alkyl)- can be synthesized by condensation of a compound of Formula Ha, with a compound of Formula V, wherein X and Y are same or different and each independently represents —NH— or —O—, in the presence or absence of a base, optionally in conjunction with a suitable catalyst (such as 4-(N,N-dimethylamino)pyridine or 1-hydroxybenzotriazole) in a suitable solvent to provide a compound of Formula VI. A compound of Formula VII (wherein X and Y are independently selected from —NH— or —O— and L is a leaving group) can be generated from the compound of Formula VI by using a suitable carbonylating reagent, for example as provided above, and then treated with a compound of Formula VIII, wherein Z is —NH— or —N(C₁₋₃ alkyl)- and n is 0 or 1, to provide the compound of Formula IV (wherein X and Y are independently selected from —NH— or —O—, Z is —NH— or —N(C₁₋₃ alkyl)- and n is 0 or 1) which then can be deprotected to yield compound of Formula I. The process can be depicted as shown in Scheme-2 below.

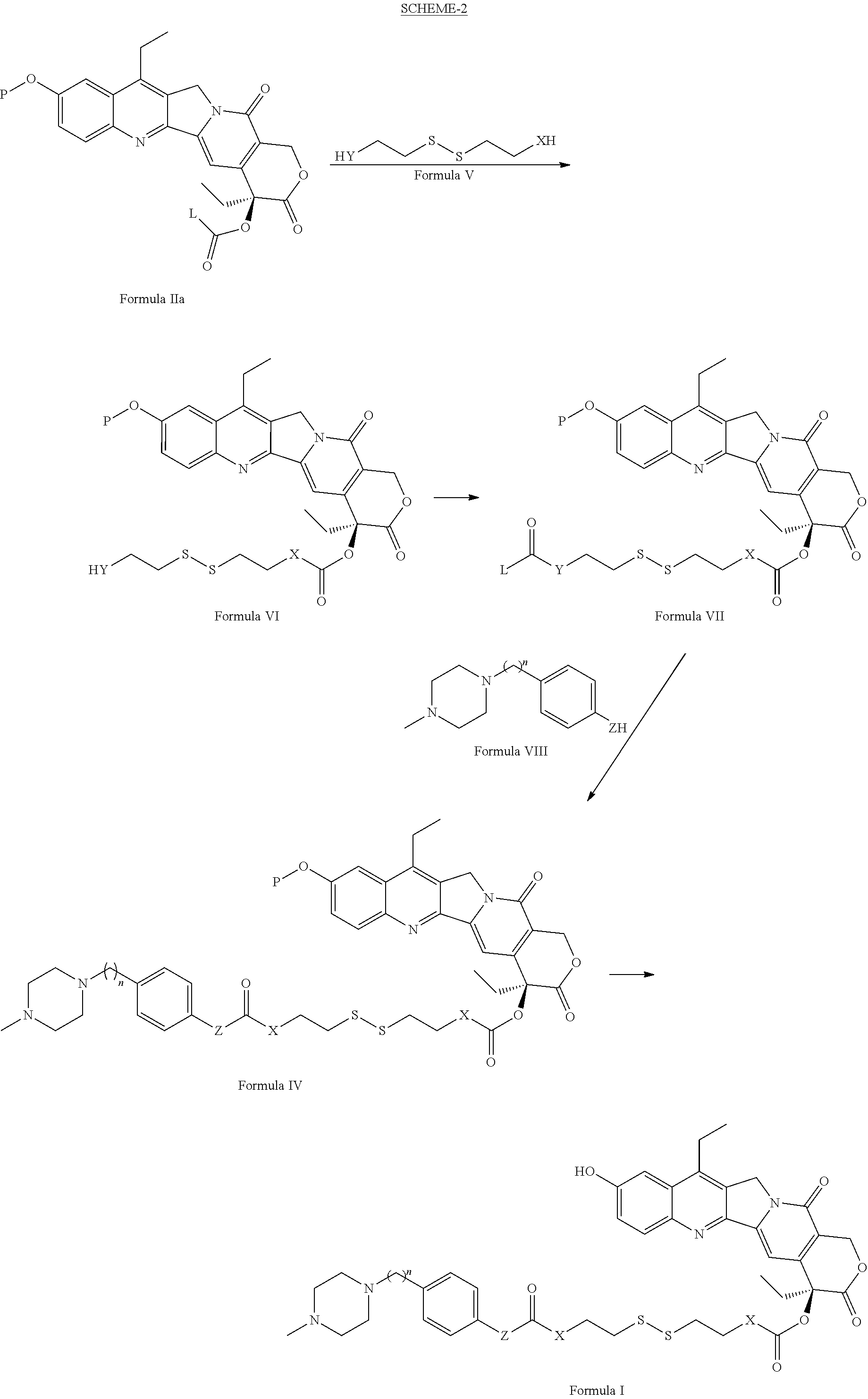

Compounds of Formula IIa and VII also can be prepared in situ without any isolation from the compound of Formula II and VI, respectively, by using a suitable carbonylating reagent commonly known for such purpose.

The compounds of Formula I, wherein X and Y are —CH₂—; Z is absent, —NH— or —N(C₁₋₃ alkyl)- and n is 0 or 1, can be synthesized by condensation of a compound of Formula II with a compound of Formula IX, wherein X and Y are —CH₂—, Z is —NH— or —N(C₁₋₃ alkyl)-, n is an integer selected from 0 or 1 and L₁ is a leaving group, in the presence or absence of inert base, optionally in conjunction with a suitable catalyst (such as 4-(N,N-dimethylamino)pyridine, 1-hydroxybenzotriazole) in an aprotic solvent to provide compound of Formula IV (wherein X and Y are —CH₂—, Z is absent, —NH— or —N(C₁₋₃ alkyl)- and n is 0 or 1) which then can be deprotected to yield a compound of Formula I. The process can be depicted as shown in Scheme-3 below.

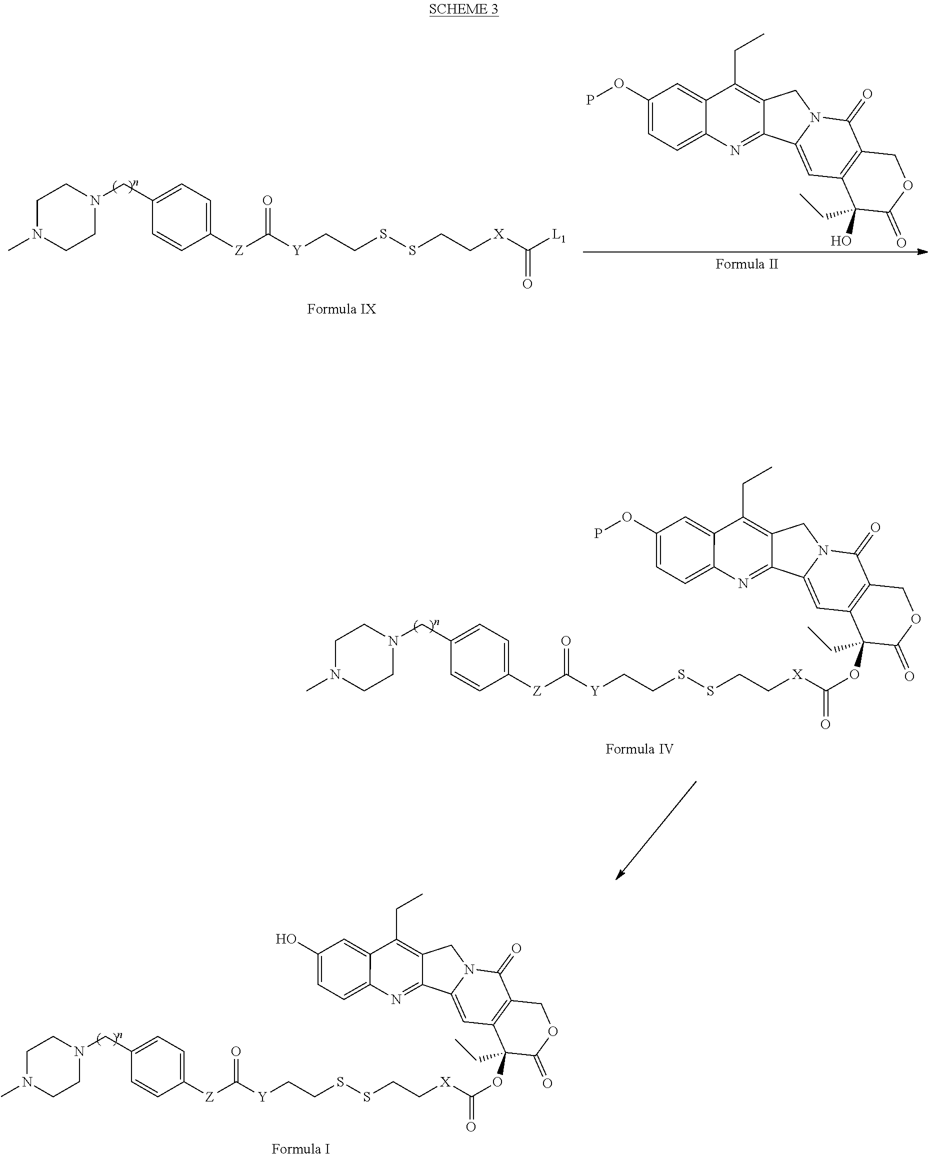

The compounds of Formula IX can be synthesized from the corresponding acids (L₁ is OH) of Formula IXc and then condensed with a compound of Formula II to generate the compound of Formula IV. Optionally, the compound of Formula IX may be prepared in situ without any isolation from the corresponding acid (L₁ is OH) of Formula IXc and then condensed with a compound of Formula II.

The compound of the Formula IX wherein L₁ is leaving group, such as a halide (L₁ is halogen), a reactive ester, a reactive anhydride, or a reactive cyclic amide can be prepared from the corresponding acid (L₁ is OH) by general methods well known to those skilled in the art. For example, compounds of Formula IX wherein L₁ is halide can be obtained by treatment of the corresponding acid (L₁ is OH) of Formula IXc with a halogenating agent, such as thionyl chloride, phosphorus pentachloride or oxalyl chloride.

Formula IX is preferably generated in situ from the corresponding acid (L₁=OH) of Formula IXc using suitable reagents in the presence or absence of an inert base, and optionally a suitable catalyst, in a suitable solvent.

The compound of Formula IXc, wherein X and Y are —CH₂—; Z is —NH— or —N(C₁₋₃ alkyl)- and n is 0 or 1, can be synthesized by condensation of compound of Formula IXa, wherein Z is —NH— or —N(C₁₋₃ alkyl)- and n is 0 or 1, with a compound of Formula IXb, wherein X and Y are —CH₂— to provide compound of Formula IXc. The process can be depicted in Scheme-3A below.

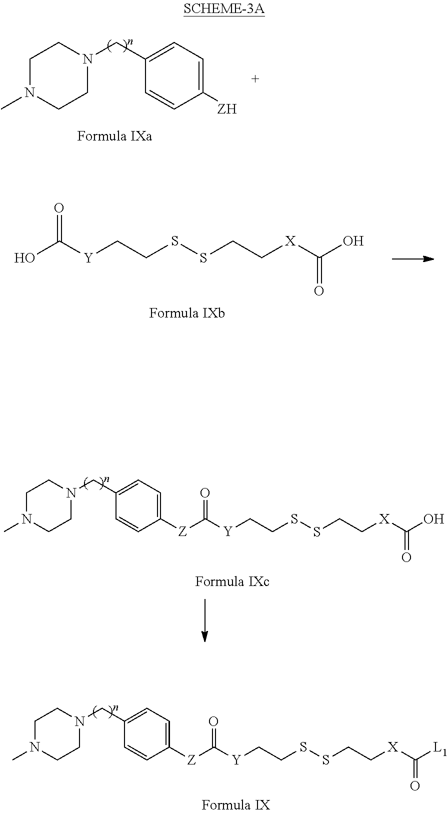

The condensation reaction can be carried out in a manner known in the art, the reaction conditions being dependent on how the acid group of formula (IXb) has been activated, usually in the presence of a suitable aprotic solvent or diluent or of a mixture thereof and, if necessary, in the presence of a condensation agent. Customary condensation agents are, for example, carbodiimides such as N,N′-diethyl-, N,N′-diisopropyl, N,N′-dicyclohexyl-or N-ethyl-N′-(3-diethylaminopropyl)carbodiimide; suitable carbonyl compounds, for example carbonyldiimidazole, or 1,2-oxazolium compounds, for example 2-ethyl-5-phenyl-1,2-oxazolium 3′-sulfonate and 2-tert-butyl-5-methyl-isoxazolium perchlorate, or a suitable acylamino compound, for example, 2-ethoxy-1-ethoxycarbonyl-1,2-dihydroquinoline. The bases normally used for aiding the condensation are either inorganic bases such as sodium or potassium carbonate, or organic bases, such as pyridine, triethylamine, N,N-diisopropylethylamine or 4-(dimethylamino)pyridine.

Similarly, the compounds of Formula I, wherein X is —CH₂— and Y is —NH— or —O—; or X is —NH—, —O— and Y is —CH₂—; and Z is absent, —NH— or —N(C₁₋₃ alkyl)- and n is 0 or 1, can be synthesized by following the process as described in Scheme 3 and Scheme 3A above by appropriately selecting the starting material, having X is —CH₂— and Y is —NH— or —O—; or X is —NH—, —O— and Y is —CH₂—; and Z is absent, —NH— or —N(C₁₋₃ alkyl)- and n is 0 or 1. For e.g. by selecting the compound of Formula IX having X is —CH₂— and Y is —NH— or —O—; or X is —NH—, —O— and Y is —CH₂—; and Z is absent, —NH— or —N(C₁₋₃ alkyl)-and n is 0 or 1, which then can be condensed with a compound of Formula II in the presence or absence of inert base, optionally in conjunction with a suitable catalyst (such as 4-(N,N-dimethylamino)pyridine, 1-hydroxybenzotriazole) in an aprotic solvent to provide compound of Formula IV (wherein X is —CH₂— and Y is —NH— or —O—; or X is —NH—, —O— and Y is —CH₂—; and Z is absent, —NH— or —N(C₁₋₃ alkyl)- and n is 0 or 1) which then can be deprotected to yield a compound of Formula I.

Alternatively, the compounds of Formula I, wherein X and Y are —CH₂— and Z is —NH— or —N(C₁₋₃ alkyl)- and n is an integer selected from 0 or 1, can be synthesized by coupling of compound of Formula II, with a compound of Formula X, wherein X and Y are —CH₂— and P is protecting group, to provide a compound of Formula XI (wherein X and Y are —CH₂—) which then may further coupled with compound of formula VIII to provide a compound of Formula IV (wherein X and Y are —CH₂—, Z is —NH— or —N(C₁₋₃ alkyl)- and n is 0 or 1), which then can be deprotected to yield compound of Formula I. The process can be depicted as shown in Scheme-4 below.

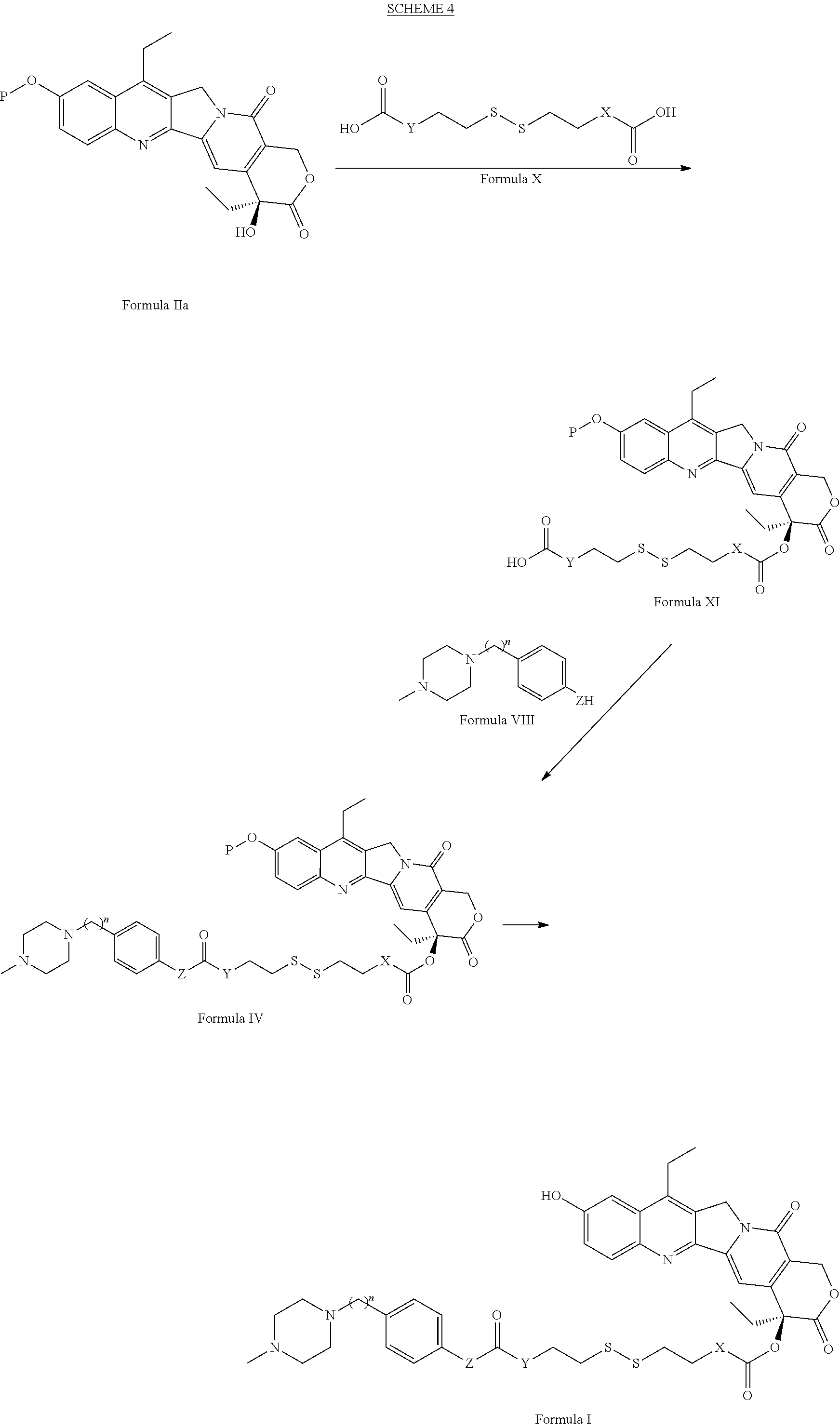

The compounds of Formula I can be converted into pharmaceutically acceptable salts of such compounds by methods known in the art, for instance, by dissolving the compound of Formula I in a suitable solvent and treating it with appropriate acid.

Table 1 provides some of the compounds of Formula I.

TABLE 1
Compounds of Formula I

| # | Structure | Chemical Name |
|---|---|---|
| I.1 | 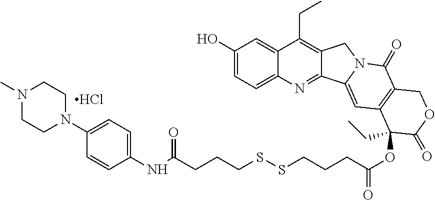 [SMILES: CCc1c2c(nc3ccc(O)cc13)-c1cc3c(c(=O)n1C2)COC(=O)[C@@]3(CC)OC(=O)CCCSSCCCC(=O)Nc1ccc(N2CCN(C)CC2)cc1 ; Cl] | 4-[3-(4-(4-Methylpiperazin-1-yl)phenyl carbamoyl)propyldisulfanyl][(4S)-4,11- diethyl-3,4,12,14-tetrahydro-9-hydroxy-1H- pyrano[3′,4′:6,7]indolizino[1,2-b]quinoline- 3,14-(4H,12H)dione-4-yl]butyrate hydrochloride |
| I.2 | 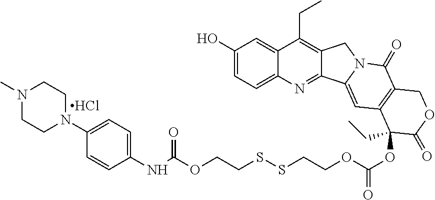 [SMILES: CCc1c2c(nc3ccc(O)cc13)-c1cc3c(c(=O)n1C2)COC(=O)[C@@]3(CC)OC(=O)OCCSSCCOC(=O)Nc1ccc(N2CCN(C)CC2)cc1 ; Cl] | 2-(2-{N-[4-(4-Methylpiperazin-1- yl)phenyl]carbamoyloxy}ethyldisulfanyl)ethyl [(4S)-4,11-diethyl-3,4,12,14-tetrahydro-9- hydroxy-1H-pyrano[3′,4′:6,7]indolizino[1,2- b]quinoline-3,14-(4H,12H)dione-4-yl] carbonate hydrochloride |
| I.3 | 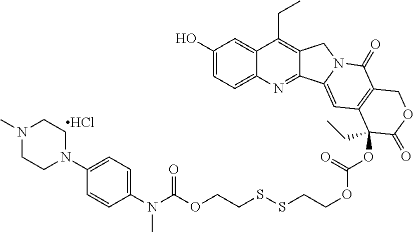 [SMILES: CCc1c2c(nc3ccc(O)cc13)-c1cc3c(c(=O)n1C2)COC(=O)[C@@]3(CC)OC(=O)OCCSSCCOC(=O)N(C)c1ccc(N2CCN(C)CC2)cc1 ; Cl] | 2-(2-{N-Methyl-N-[4-(4-methylpiperazin-1- yl)phenyl] carbamoyloxy}ethyldisulfanyl)ethyl [(4S)- 4,11-diethyl-3,4,12,14-tetrahydro-9-hydroxy- 1H-pyrano[3′,4′:6,7]indolizino[1,2- b]quinoline-3,14-(4H,12H)dione-4-yl] carbonate hydrochloride |
| I.4 | 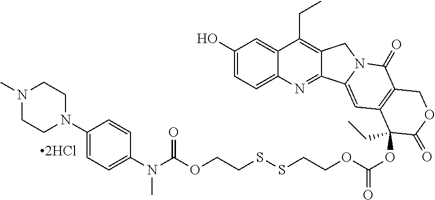 [SMILES: CCc1c2c(nc3ccc(O)cc13)-c1cc3c(c(=O)n1C2)COC(=O)[C@@]3(CC)OC(=O)OCCSSCCOC(=O)N(C)c1ccc(N2CCN(C)CC2)cc1 ; "·2HCl"] | 2-(2-{N-Methyl-N-[4-(4-methylpiperazin-1- yl)phenyl]carbamoyloxy}ethyldisulfanyl)ethyl [(4S)-4,11-diethyl-3,4,12,14-tetrahydro-9- hydroxy-1H-pyrano[3′,4′:6,7]indolizino[1,2- b]quinoline-3,14 -(4H,12H)dione-4-yl] carbonate dihydrochloride |
| I.5 | 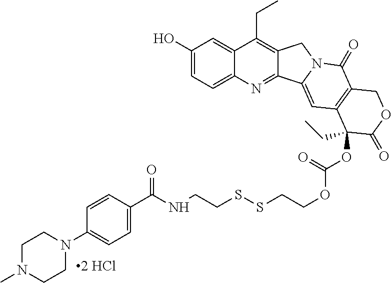 [SMILES: CCc1c2c(nc3ccc(O)cc13)-c1cc3c(c(=O)n1C2)COC(=O)[C@@]3(CC)OC(=O)OCCSSCCNC(=O)c1ccc(N2CCN(C)CC2)cc1] | 2-(2-{4-[4-Methylpiperazin-1- yl]benzoylamino}ethyldisulfanyl) ethyl [(4S)- 4,11-diethyl-3,4,12,14-tetrahydro-9-hydroxy- 1H-pyrano[3′,4′:6,7]indolizino[1,2- b]quinoline-3,14-(4H,12H)dione-4- yl]carbonate dihydrochloride |
| I.6 | 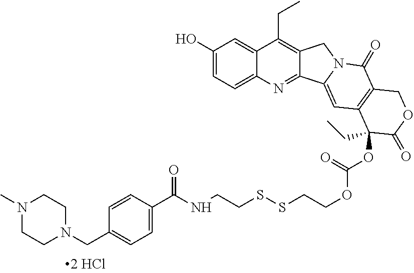 [SMILES: CCc1c2c(nc3ccc(O)cc13)-c1cc3c(c(=O)n1C2)COC(=O)[C@@]3(CC)OC(=O)OCCSSCCNC(=O)c1ccc(CN2CCN(C)CC2)cc1] | 2-(2-{4-[4-Methylpiperazin-1- ylmethyl]benzoylamino}ethyl disulfanyl)ethyl [(4S)-4,11-diethyl-3,4,12,14-tetrahydro-9- hydroxy-1H-pyrano[3′,4′:6,7]indolizino[1,2- b]quinoline-3,14-(4H,12H)dione-4-yl] carbonate dihydrochloride |
| I.7 | 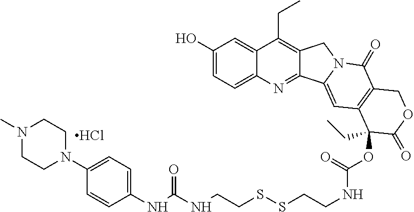 [SMILES: CCc1c2c(nc3ccc(O)cc13)-c1cc3c(c(=O)n1C2)COC(=O)[C@@]3(CC)OC(=O)NCCSSCCNC(=O)Nc1ccc(N2CCN(C)CC2)cc1 ; Cl] | [2-(2-{3-[4-(4-Methylpiperazin-1- yl)phenyl]ureido}ethyldisulfanyl)ethyl][(45)- 4,11-diethyl-3,4,12,14-tetrahydro-9-hydroxy- 1H-pyrano[3′,4′:6,7]indolizino[1,2- b]quinoline-3,14-(4H,12H )dione-4-yl] carbamate hydrochloride. |

The present invention is further illustrated in detail with reference to the following examples. It is desired that the examples be considered in all respect as illustrative and are not intended to limit the scope of the claimed invention.

### Experimental

All solvents and reagents were used as obtained from commercial sources unless otherwise indicated. ¹H-NMR spectra were recorded with a Bruker Bio spin AG-500 operating at 500 MHz in deuterated DMSO solvent. The mass spectra were recorded using Waters Acquity QDa.

### EXAMPLE 1

### 7-Ethyl-10-(tert-butoxycarbonyoxy) camptothecin

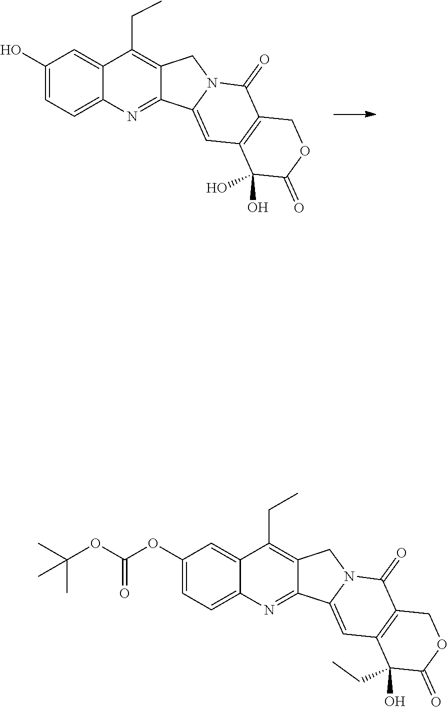

Di-tert-butyl dicarbonate (22.08 mL, 99.3 mmol) and pyridine (121.0 ml, 1.53 mol) were added to the suspension of 7-ethyl-10-hydroxycamptothecin (30.0 g, 76.4 mmol) in dichloromethane (600 mL). The suspension was stirred overnight at 25-30° C. The reaction mixture was filtered and the filtrate was washed with 0.5 N hydrochloric acid followed by saturated sodium bicarbonate solution. The dichloromethane layer was dried and concentrated in vacuo to yield the compound as a light yellow solid (26.0 g).

### EXAMPLE 2

### 4-[3-a4S)-9-tert-Butoxycarbonyloxy-4,11-diethyl-3,4,12,14-tetrahydro-1H-pyrano[3′,4′:6,7]indolizino[1,2-b]quinoline-3,14-(4H,12H)dione-4-yl oxycarbonyl)propyldisulfanyl]butyric acid

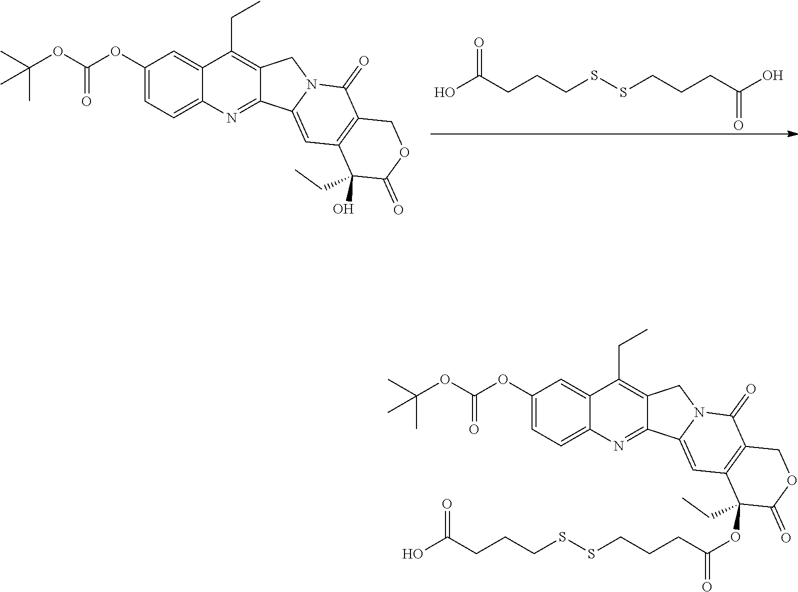 [SMILES: CCc1c2c(nc3ccc(OC(=O)OC(C)(C)C)cc13)-c1cc3c(c(=O)n1C2)COC(=O)[C@]3(O)CC ; O=C(O)CCCSSCCCC(=O)O ; CCc1c2c(nc3ccc(OC(=O)OC(C)(C)C)cc13)-c1cc3c(c(=O)n1C2)COC(=O)[C@@]3(CC)OC(=O)CCCSSCCCC(=O)O]

1-Ethyl-3-(3-dimethylaminopropyl)carbodiimide (EDC) hydrochloride (1.17 g, 6.09 mmol) was added to a stirred solution of 4,4′-dithiodibutyric acid (2.9 g, 12.2 mmol) in 60 ml of dichloromethane at 10-15° C. The mixture was stirred at 20-25° C. After 0.5 hr, 7-ethyl-10-(tert-butyloxycarbonyoxy)camptothecin (3 g, 6.09 mmol) and 4-dimethylaminopyridine (0.491 g, 4.01 mmol) were added to the reaction mixture, and stirring was continued for 3 hrs. The reaction mixture was quenched with water and the dichloromethane layer was separated, washed with water, dried and concentrated in vacuo. The residue was purified by column chromatography on silica gel (75% ethyl acetate in n-hexane) to yield the title compound as a light yellow solid.

### EXAMPLE 3

### 4-[3-(4-(4-Methylpiperazin-1-yl)phenylcarbamoyl)propyl disulfanyl][(4S)-9-tert-butyloxycarbonyloxy-4,11-diethyl-3,4,12,14-tetrahydro-1H-pyrano[3′,4′:6,7]indolizino[1,2-b]quinoline-3,14-(4H,12H) dione-4-yl] butyrate

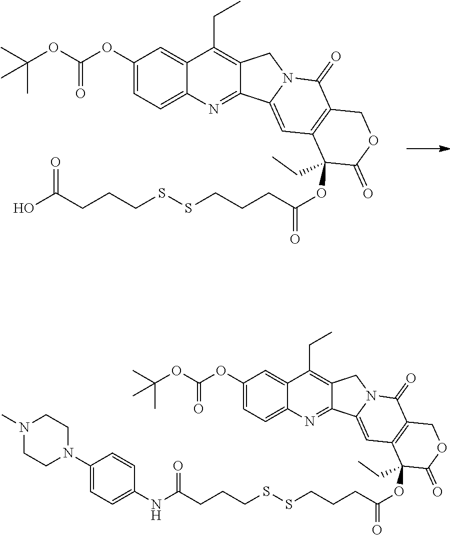 [SMILES: CCc1c2c(nc3ccc(OC(=O)OC(C)(C)C)cc13)-c1cc3c(c(=O)n1C2)COC(=O)[C@@]3(CC)OC(=O)CCCSSCCCC(=O)O ; CCc1c2c(nc3ccc(OC(=O)OC(C)(C)C)cc13)-c1cc3c(c(=O)n1C2)COC(=O)[C@@]3(CC)OC(=O)CCCSSCCCC(=O)Nc1ccc(N2CCN(C)CC2)cc1]

EDC hydrochloride (0.64 g, 3.36 mmol) was added to a stirred solution of 4-[3-((4S)-9-tert-butoxycarbonyloxy-4,11-diethyl-3,4,12,14-tetrahydro-1H-pyrano [3′,4′:6,7] indolizino[1,2-b]quinoline-3,14-(4H,12H)dione-4-yl oxycarbonyl)propyl disulfanyl]butyric acid (1.6 g, 2.24 mmol) in 40 ml of dichloromethane at 10-15° C. The mixture was stirred at 20-25° C. After 0.5 hr, 4-(4-methylpiperazin-1-yl)phenylamine (0.514 g, 2.68 mmol) and 4-dimethylaminopyridine (0.028 g, 0.22 mmol) were added to the reaction mixture, and stirring was continued for 3 hrs. The reaction mixture was quenched with water and the dichloromethane layer was separated, washed with water, dried and concentrated in vacuo to yield a residue. The residue was purified by column chromatography on silica gel (5% methanol in dichloromethane) to yield the title compound as a light yellow solid.

### EXAMPLE 4

### 4-[3-(4-(4-Methylpiperazin-1-yl)phenyl carbamoyl)propyl disulfanyl][(4S)-4,11-diethyl-3,4,12,14-tetrahydro-9-hydroxy-1H-pyran [3′,4′:6,7]indolizino[1,2-b]quinoline-3,14-(4H,12H)dione-4-yl]butyrate hydrochloride (Compound I.1)

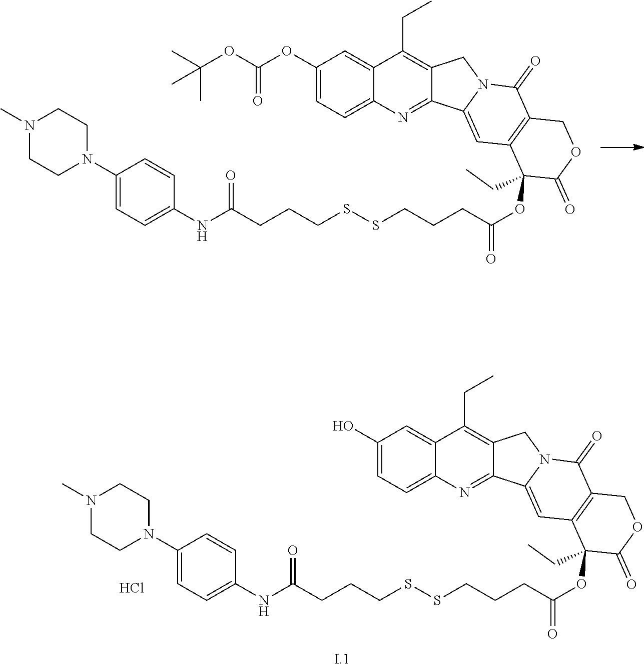 [SMILES: CCc1c2c(nc3ccc(OC(=O)OC(C)(C)C)cc13)-c1cc3c(c(=O)n1C2)COC(=O)[C@@]3(CC)OC(=O)CCCSSCCCC(=O)Nc1ccc(N2CCN(C)CC2)cc1 ; CCc1c2c(nc3ccc(O)cc13)-c1cc3c(c(=O)n1C2)COC(=O)[C@@]3(CC)OC(=O)CCCSSCCCC(=O)Nc1ccc(N2CCN(C)CC2)cc1 ; Cl]

Piperidine (0.172 g, 2.03 mmol) was added to a stirred solution of 4-[3-(4-(4-methylpiperazin-1-yl)phenylcarbamoyl)propyldisulfanyl] [(4S)-9-tert-butyloxy carbonyloxy-4,11-diethyl-3,4,12,14-tetrahydro-1H-pyrano[3′,4′:6,7]indolizino [1,2-b] quinoline-3,14-(4H,12H)dione-4-yl] butyrate (0.9 g, 1.01 mmol) in 15 ml of acetone at 20-25° C. and stirring was continued for 6 hrs. The reaction mixture was concentrated and the residue was stirred with diethyl ether. The solid was filtered, washed with diethyl ether and purified by column chromatography on silica gel (5 to 20% methanol in dichloromethane). The pure solid was dissolved in mixture of dichloromethane-methanol and treated with 1 molar equivalent of hydrochloric acid at 10-15° C. The solution was concentrated and the residue was stirred with acetone. The resulting solid was filtered, washed with acetone and dried to yield the title compound as a light yellow solid.

H¹NMR (500 MHz, DMSO-d6, δ ppm): 0.97(t, J=7.40 Hz, 3H), 1.33(t, J=7.6 Hz, 3H), 1.93-2.0(m, 4H), 2.14-2.20(m, 2H), 2.39(t, J=7.28 Hz, 2H), 2.71(t, J=7.24 Hz, 2H), 2.76-2.82(m, 4H), 2.87(d, J=4.31 Hz, 3H), 3.01(t, J-11.77 Hz, 2H), 3.12-3.19(m, 4H), 3.52(d, J=13.5 Hz, 2H), 3.76(d, J=13.22 Hz, 2H), 5.34(s, 2H), 5.54(s, 2H), 6.97(d, J=9.09 Hz, 2H), 6.99(s, 1H), 7.46-7.51(m, 4H), 8.07(d, J=9.13 Hz, 2H), 9.81(s, 1H), 10.43(s, 1H). Mass (ES+, m/z): 786.30 (M+H)⁺

### EXAMPLE 5

### 2-(2-Hydroxyethyldisulfanyl)ethyl [4S)-9-tert-butyloxycarbonyl oxy-4,11-diethyl-3,4,12,14-tetrahydro-1H-pyrano[3′,4′:6,7]indolizino[1,2-b]quinoline-3,14-(4H,12H)dione-4-yl] carbonate

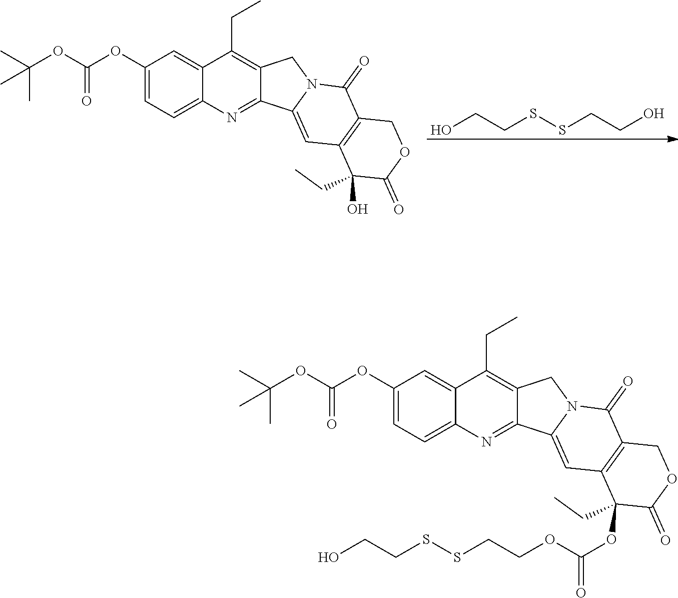

Triphosgene (1.44 g, 4.87 mmol) was added to a stirred mixture of 7-ethyl-10-(tert-butyloxycarbonyoxy)camptothecin (tert-butyloxycarbonyoxy)camptothecin (6 g, 12.18 mmol) and 4-dimethylaminopyridine (4.46 g, 36.5 mmol) in dichloromethane (90 mL) at 10-15° C. The mixture was stirred under a blanket of nitrogen at 20-25° C. After 0.5 hr, 2,2′-dithiodiethanol (3.75 g, 24.36 mmol) was added to the reaction mixture, and stirring was continued for 3 hrs. The reaction mixture was quenched with water and the dichloromethane layer was separated, washed with water, dried and concentrated in vacuo to yield a residue. The residue was purified by column chromatography on silica gel (75% ethyl acetate in n-hexane) to yield the compound as a light yellow solid.

### EXAMPLE 6

### 2-(2-{N-[4-(4-Methylpiperazin-1-yl)phenyl]carbamoyloxy} ethyldisulfanyl)ethyl [(4S)-9-tert-butyloxycarbonyloxy-4,11-diethyl-3,4,12,14-tetrahydro-1H-pyrano[3′,4′:6,7]indolizino[1,2-b]quinoline-3,14-(4H,12H)dione-4-yl] carbonate

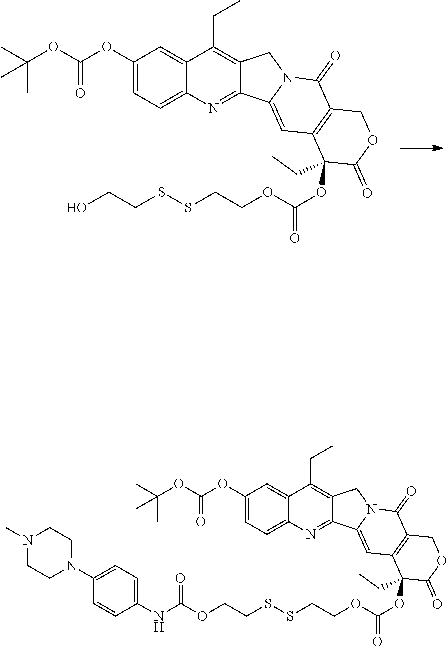 [SMILES: CCc1c2c(nc3ccc(OC(=O)OC(C)(C)C)cc13)-c1cc3c(c(=O)n1C2)COC(=O)[C@@]3(CC)OC(=O)OCCSSCCO ; CCc1c2c(nc3ccc(OC(=O)OC(C)(C)C)cc13)-c1cc3c(c(=O)n1C2)COC(=O)[C@@]3(CC)OC(=O)OCCSSCCOC(=O)Nc1ccc(N2CCN(C)CC2)cc1]

Triphosgene (0.32 g, 1.07 mmol) was added to a stirred mixture of 2-(2-hydroxyethyldisulfanyl)ethyl [(45)-9-tert-butyloxycarbonyloxy-4,11-diethyl-3,4,12,14-tetrahydro-1H-pyrano[3′,4′:6,7]indolizino[1,2-b]quinoline-3,14-(4H,12H)dione-4-yl] carbonate (1.8 g, 2.67 mmol) and 4-dimethylaminopyridine (0.98 g, 8.01 mmol) in dichloromethane (60 mL) at 15-20° C. The mixture was stirred under a blanket of nitrogen at 20-25° C. After 0.5 hr, 4-(4-methylpiperazin-1-yl)phenylamine (0.51 g, 2.66 mmol) was added in the reaction mixture, and stirring was continued for 3 hrs. The reaction mixture was quenched with water and the dichloromethane layer was separated, washed with water, dried and concentrated in vacuo to yield a residue. The residue was purified by column chromatography on silica gel (5% methanol in dichloromethane) to yield the compound as a light yellow solid.

### EXAMPLE 7

### 2-(2-{N-[4-(4-Methylpiperazin-1-yl)phenyl]carbamoyloxy} ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate (Compound I.2)

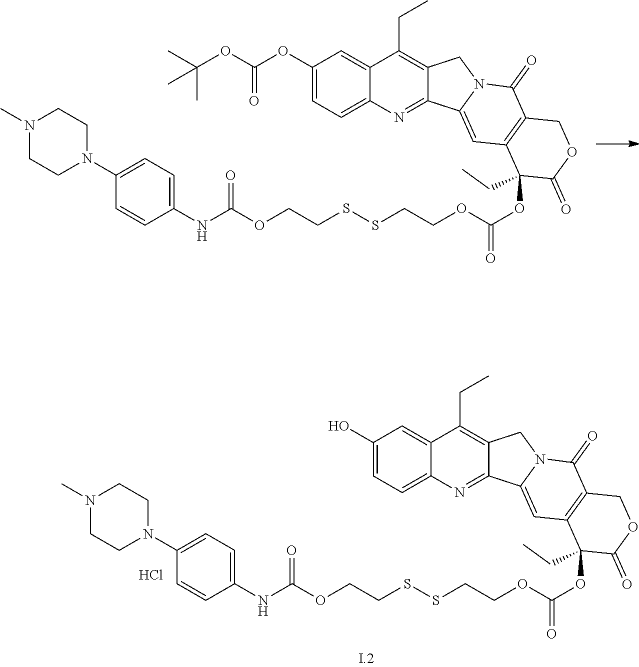 [SMILES: CCc1c2c(nc3ccc(OC(=O)OC(C)(C)C)cc13)-c1cc3c(c(=O)n1C2)COC(=O)[C@@]3(CC)OC(=O)OCCSSCCOC(=O)Nc1ccc(N2CCN(C)CC2)cc1 ; CCc1c2c(nc3ccc(O)cc13)-c1cc3c(c(=O)n1C2)COC(=O)[C@@]3(CC)OC(=O)OCCSSCCOC(=O)Nc1ccc(N2CCN(C)CC2)cc1 ; Cl]

Piperidine (0.183 g, 2.15 mmol) was added to a stirred solution of carbonic acid 2-(2-{N-[4-(4-methylpiperazin-1-yl)phenyl]carbamoyloxy} ethyldisulfanyl)ethyl [(4S)-9-tert-butyloxycarbonyloxy-4,11-diethyl-3,4,12,14-tetrahydro-1H-pyrano[3′,4′:6,7] indolizino[1,2-b]quinoline-3,14-(4H,12H)dione-4-yl] carbonate (0.95 g, 1.07 mmol) in 10 ml of acetone at 20-25° C. and stirring was continued for 3 hrs. The reaction mixture was concentrated and residue was stirred with diisopropyl ether. The solid was filtered, washed with diisopropyl ether and purified by column chromatography on silica gel (5 to 15% methanol in dichloromethane). The pure solid was dissolved in mixture of dichloromethane-methanol and treated with 1 molar equivalent of hydrochloric acid at 10-15° C. The solution was concentrated and the residue was stirred with acetone. The resulting solid was filtered, washed with acetone and dried to yield the title compound as a light yellow solid.

¹H-NMR (500 MHz, DMSO-d6, δ ppm): 0.96(t, J=7.37 Hz, 3H), 1.33(t, J=7.57 Hz, 3H), 2.19-2.24(m, 2H), 2.86(d, J=4.6 Hz, 3H), 3.02-3.22 (m, 12H), 3.74(d, J-13.1 Hz, 2H), 4.29(t, J=6.12 Hz, 2H), 4.39(t, J=5.6 Hz, 2H), 5.35(s, 2H), 5.57(s, 2H), 6.97(d, J=9.0 Hz, 2H), 7.02(s, 1H), 7.37(d, J=6.76 Hz, 2H), 7.46-7.48(m, 2H), 8.08(d, J=9.86 Hz, 2H), 9.52(br-s, 1H), 10.44(br-s, 1H). Mass (ES+, m/z): 790.27.

### EXAMPLE 8

### Acetic acid 2-(2-{methyl-[4-(4-methylpiperazin-1-yl)phenyl] carbamoyloxy}ethyldisulfanyl)ethyl ester

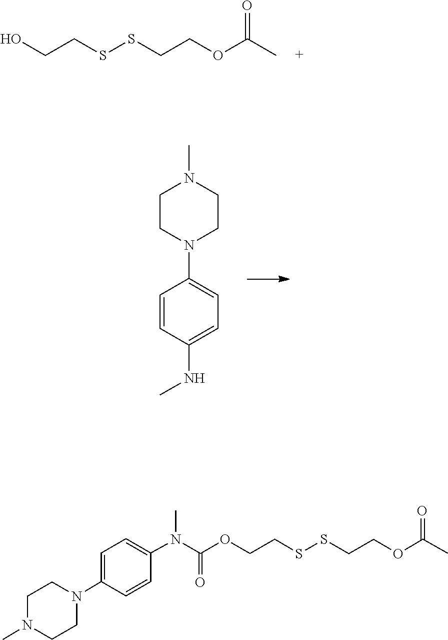 [SMILES: CC(=O)OCCSSCCO ; CNc1ccc(N2CCN(C)CC2)cc1 ; CC(=O)OCCSSCCOC(=O)N(C)c1ccc(N2CCN(C)CC2)cc1]

Triphosgene (2.31 g, 0.008 mol) was added portion wise to a solution of N-methyl-4-(4-methylpiperazin-1-yl)aniline (4 g, 0.019 mol) in dichloromethane (40 mL) at 25-30° C., and stirring was continued for 1.5 hrs. The reaction mixture was quenched with sat. sodium bicarbonate solution (40 mL). The product was extracted with dichloromethane. The dichloromethane layer was dried over anhydrous sodium sulfate and concentrated to give N-methyl-N-[4-(4-methylpiperazin-1-yl)phenyl]carbamoyl chloride as a brown solid. A solution of N-methyl-N-[4-(4-methylpiperazin-1-yl)phenyl]carbamoyl chloride in acetonitrile (20 mL) was added dropwise to a mixture of acetic acid 2-(2-hydroxyethyldisulfanyl)ethyl ester (4 g, 0.020 mol) in acetonitrile (20 mL), triethyl amine (5.47 ml, 0.0.039 mol), and 4-dimethylaminopyridine(1.18 g, 0.010 mol) at 15-20° C. The reaction mixture was stirred at 85° C. for 8-9 hrs. Reaction mixture was cooled to room temperature and quenched by demineralised (DM) water and product was extracted with ethyl acetate. The ethyl acetate layer was washed with DM water, dried over anhydrous sodium sulfate and concentrated under vacuum to yield a residue. The residue was purified by column chromatography (5% methanol in ethyl acetate) to give title compound as a brown liquid (3.7 g).

### EXAMPLE 9

### Methyl-[4-(4-methylpiperazin-1-yl)phenyl]carbamic acid 2-(2-hydroxyethyldisulfanyl)ethyl ester

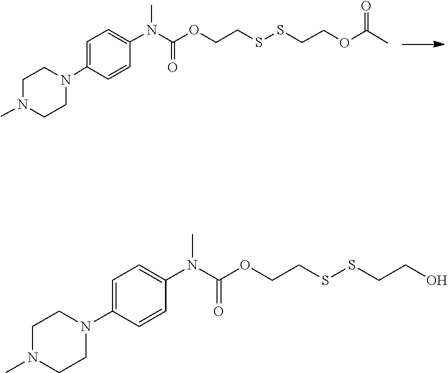

To a solution of acetic acid 2-(2-{methyl-[4-(4-methylpiperazin-1-yl)phenyl]carbamoyloxy}ethyldisulfanyl)ethyl ester (3.5 g, 0.008 mol) in methanol (14 mL) was added p-toluenesulfonic acid monohydrate (4.33g, 0.025 mol) at 25-30° C., and stirring was continued for 6 hrs. The reaction mixture was diluted with dichloromethane (35 mL), followed by saturated sodium bicarbonate solution (28 mL). The organic layer was separated, dried over sodium sulphate and concentrated under vacuum. The resulting residue was purified by column chromatography (5-10% methanol in ethyl acetate) to give title compound as yellowish brown solid (2.6 g).

### EXAMPLE 10

### 2-(2-{N-Methyl-N-[4-(4-methylpiperazin-1-yl)phenyl]carbamoyloxy}ethyl disulfanyl)ethyl[(4S)-9-tert-butyloxycarbonyloxy-4,11-diethyl-3,4,12,14-tetrahydro-1H-pyrano[3′,4′:6,7]indolizino[1,2-b]quinoline-3,14-(4H,12H)dione-4-yl] carbonate

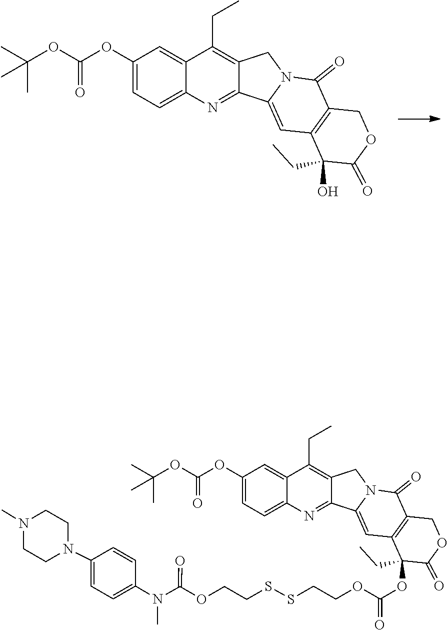 [SMILES: CCc1c2c(nc3ccc(OC(=O)OC(C)(C)C)cc13)-c1cc3c(c(=O)n1C2)COC(=O)[C@]3(O)CC ; CCc1c2c(nc3ccc(OC(=O)OC(C)(C)C)cc13)-c1cc3c(c(=O)n1C2)COC(=O)[C@@]3(CC)OC(=O)OCCSSCCOC(=O)N(C)c1ccc(N2CCN(C)CC2)cc1]

Triphosgene (1.57 g, 5.28 mmol) was added to a stirred mixture of 7-ethyl-10-(tert-butyloxycarbonyoxy)camptothecin (6.5 g, 13.2 mmol) and 4-dimethylaminopyridine (4.83 g, 39.6 mmol) in dichloromethane (65 mL) at 20-25° C. The mixture was stirred under a blanket of nitrogen at 20-25° C. After 0.5 hr, methyl[4-(4-methylpiperazin-1-yl)phenyl]carbamic acid 2-(2-hydroxyethyl disulfanyl)ethyl ester (4.57 g, 11.9 mmol) was added to the reaction mixture, and stirring was continued for 3 hrs. The reaction mixture was quenched with water and the dichloromethane layer was separated, washed with water, dried and concentrated in vacuo. The resulting residue was purified by column chromatography on silica gel (5% methanol in dichloromethane) to yield the compound as a light yellow solid.

### EXAMPLE 11

### 2-(2-{N-Methyl-N-[4-(4-methylpiperazin-1-yl)phenyl]carbamoyloxy}ethyl disulfanyl)ethyl[(4S)-4,11-diethyl-3,4,12,14-tetrahydro-9-hydroxy-1H-pyrano[3′,4′:6,7] indolizino[1,2-b]quinoline-3,14-(4H,12H)dione-4-yl]carbonate hydrochloride (Compound I.3)

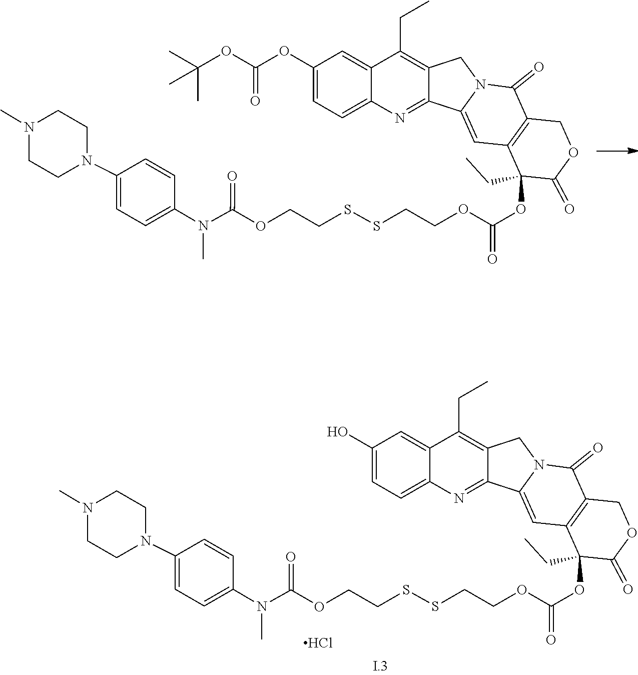 [SMILES: CCc1c2c(nc3ccc(OC(=O)OC(C)(C)C)cc13)-c1cc3c(c(=O)n1C2)COC(=O)[C@@]3(CC)OC(=O)OCCSSCCOC(=O)N(C)c1ccc(N2CCN(C)CC2)cc1 ; CCc1c2c(nc3ccc(O)cc13)-c1cc3c(c(=O)n1C2)COC(=O)[C@@]3(CC)OC(=O)OCCSSCCOC(=O)N(C)c1ccc(N2CCN(C)CC2)cc1 ; Cl]

Piperidine (1.22 g, 14.4 mmol) was added to a stirred solution of 2-(2-{N-methyl-N-[4-(4-methylpiperazin-1-yl)phenyl]carbamoyloxy}ethyldisulfanyl)ethyl [(45)-9-tert-butyloxycarbonyloxy-4,11-diethyl-3,4,12,14-tetrahydro-1H-pyrano[3′,4′:6,7]indolizino [1,2-b]quinoline-3,14-(4H,12H)dione-4-yl] carbonate (6.5 g, 7.19 mmol) in 65 ml of acetone at 20-25° C. and stirring was continued for 4hrs. The reaction mixture was concentrated and residue was stirred with diisopropyl ether. The resulting solid was filtered, washed with diisopropyl ether and purified by column chromatography on silica gel (10% methanol in dichloromethane).

The pure solid (4.32 g, 0.005 mol) was dissolved in a mixture of dichloromethane-methanol and treated with hydrochloric acid in methanol (10.1 ml, 0.007 mol) at 10-15° C. The solution was concentrated and the residue was stirred with acetone. The resulting solid was filtered, washed with acetone and dried to yield the title compound (4.0 g).

¹H-NMR (500 MHz, DMSO-d6, δ ppm): 0.96(t, J=7.37 Hz, 3H), 1.34(t, J=7.57 Hz, 3H), 2.16-2.26(m, 2H), 2.87(d, J=4.6 Hz, 3H), 2.96-3.20(m, 10H), 3.16(s, 3H), 3.52(d, J=11.17 Hz, 2H), 3.83(d, J=12.91 Hz, 2H), 4.22(s, 2H), 4.35(s, 2H), 5.35(s, 2H), 5.57(s, 2H), 6.98(d, J=8.79 Hz, 2H), 7.02(s, 1H), 7.17(d, J=8.43 Hz, 2H), 7.47(s, 1H), 7.48(d, J=7.07 Hz, 1H), 8.06(d, J=9.84 Hz, 1H), 10.45(bs, 1H), 10.52(br-s, 1H). Mass (ES+, m/z): 803.97.

### Chloride Content (by Ion Chromatography): 4.56%

The chloride content was determined by using ion chromatography with a Dionex ICS-3000 (Thermo Scientific) using the following method:

### Mobile Phase

An accurately weighed 2.4150 g of sodium hydroxide (50% solution for ion chromatography) was transferred into a 2000 ml volumetric flask. The sodium hydroxide was dissolved in and diluted up to the mark with milli-Q-water (15 mM NaOH solution)

Water was used as the diluent.

### Standard Stock Solution Preparation

A sufficient quantity of sodium chloride was dried at 105° C. for approximately 30 min.

An accurately weighed 123.00 mg of previously dried sodium chloride was transferred into a 100 ml volumetric flask. About 50 ml diluent was added, and the solution was sonicated to dissolve the content, diluted up to the mark with diluent and mixed well. This solution contains the equivalent of 750 μg/mL of chloride.

### Standard Solution Preparation

An aliquot of 1.0 mL of standard stock solution was transferred into a 10 mL volumetric flask, diluted up to the mark with diluent and mixed well. This solution contains the equivalent of 75 μg/mL of chloride.

### Test Solution Preparation

An accurately weighed 14.96 mg of sample was transferred into a 10 ml of volumetric flask. About 5 ml of diluent was added, and the solution was sonicated to dissolve the content. The mixture was diluted up to the mark with diluent and mixed well.

### Instrumental conditions:

A suitable Ion-chromatography was connected to a conductivity detector with the following conditions.

| Column | Ion Pac AG11 HC (4.0 × 50 | |
|---|---|---|
| | mm) + Ion Pac AS11 HC | |
| | (4.0 × 250 mm) (Make: Dionex) | |
| Flow rate | 1.5 | ml/min |
| Column Temperature | 35° | C. |
| Suppressor | AERS 400-4 mm | |
| Suppressor current | 56 | mA |
| Detector | Conductivity detector | |
| Cell temperature | 35° | C. |
| Compartment temperature | 30° | C. |
| Injection volume | 10 | μl |
| Run time | 20 | min |

| | |
|---|---|
| Retention time | About 3.75 min for chloride |

### Procedure

The chromatographic system was set to the instrumental conditions described above and equilibrated at least for 60 min. Two to three replicate injections of diluent was injected for system saturation. 10 μl of diluent as a blank was injected and the chromatogram was recorded up to 20min. 10 μl of standard solution was injected in six replicates and the chromatograms was recorded up to 20 min. 10 μl of test solution was injected and the chromatogram was recorded up to 20 min. The retention time of chloride was about 3.75 min. The chloride content was calculated by an external standard method.

### EXAMPLE 12

### 2-(2-{N-Methyl-N-[4-(4-methylpiperazin-1-yl)phenyl] carbamoyloxy}ethyl disulfanyl)ethyl[(4S)-4,11-diethyl-3,4,12,14-tetrahydro-9-hydroxy-1H-pyrano[3′,4′:6,7] indolizino[1,2-b]quinoline-3,14-(4H,12H)dione-4-yl]carbonate dihydrochloride (Compound I.4)

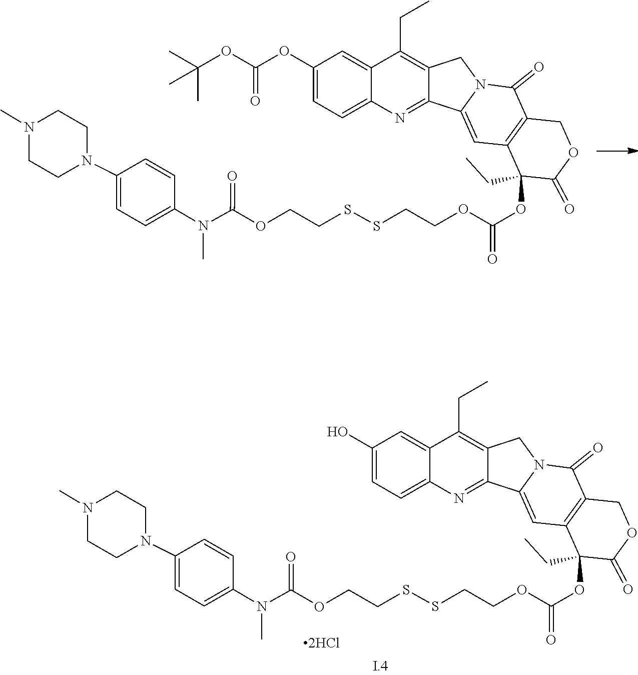 [SMILES: CCc1c2c(nc3ccc(OC(=O)OC(C)(C)C)cc13)-c1cc3c(c(=O)n1C2)COC(=O)[C@@]3(CC)OC(=O)OCCSSCCOC(=O)N(C)c1ccc(N2CCN(C)CC2)cc1 ; CCc1c2c(nc3ccc(O)cc13)-c1cc3c(c(=O)n1C2)COC(=O)[C@@]3(CC)OC(=O)OCCSSCCOC(=O)N(C)c1ccc(N2CCN(C)CC2)cc1 ; "·2HCl"]

Piperidine (4.33 g, 50.9 mmol) was added to a stirred solution of 2-(2-{N-methyl-N-[4-(4-methylpiperazin-1-yl)phenyl]carbamoyloxy}ethyldisulfanyl)ethyl [(4S)-9-tert-butyloxy carbonyloxy-4,11-diethyl-3,4,12,14-tetrahydro-1H-pyrano[3′,4′:6,7]indolizino[1,2-b] quinoline-3,14-(4H,12H)dione-4-yl]carbonate (23.0 g, 25.4 mmol) in 230 ml of acetone at 20-25° C. and stirring was continued for 4 hrs. The reaction mixture was concentrated and the residue was stirred with diisopropyl ether. The resulting solid was filtered, washed with diisopropyl ether and purified by column chromatography on silica gel (10% methanol in dichloromethane). The pure solid (15.5 g, 0.019 mol) was dissolved in hydrochloric acid in methanol (92 mL, 0.067 mol) and dichloromethane (80 mL) at 25-30° C. Clear solution was added dropwise to the diisopropyl ether at room temperature. The resulting solid was filtered and washed with diisopropyl ether and dried to give title compound (14.5 g).

¹H-NMR (500 MHz, DMSO-d6, δ ppm): 0.96(t, J=7.37 Hz, 3H), 1.33(t, J=7.57 Hz, 3H), 2.16-2.26(m, 2H), 2.85(d, J=4.62 Hz, 3H), 2.95-3.03(m, 4H), 3.08-3.21(m, 9H), 3.51(d, J-10.90 Hz, 2H), 3.82(d, J-12.05 Hz, 2H), 4.22(s, 2H), 4.35(s, 2H), 5.35(s, 2H), 5.57(S, 2H), 6.98(d, J=8.76 Hz, 2H), 7.05(s, 1H), 7.16(d, J=8.38 Hz, 2H), 7.48(s, 1H), 7.49(d, J=7.89 Hz, 1H), 8.07(d, J=9.56 Hz, 1H), 10.52(br-s, 1H), 10.88(br-s, 1H). Mass (ES+, m/z): 803.86. Chloride Content (by ion chromatography): 7.54%. The chloride content was determined by the method as described above in the specification.

### Comparison of Solubility of Compound I.4 in Water with SN-38

| Compound I.4 | SN-38 |
|---|---|
| Solubility: 100 mg/11 ml in water. | Practically insoluble in water |

### EXAMPLE 13

### N-[2-(2-Hydroxyethyldisulfanyl)ethyl]-4-(4-methylpiperazin-1-ylmethyl) benzamide

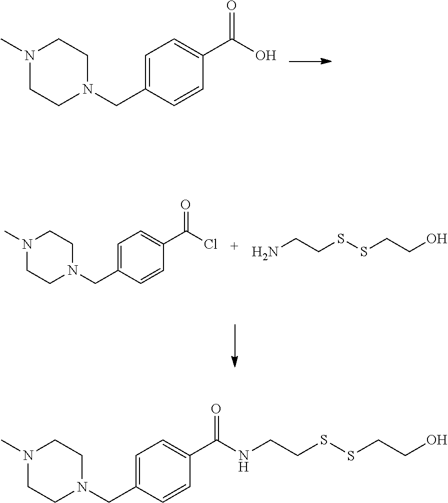 [SMILES: CN1CCN(Cc2ccc(C(=O)O)cc2)CC1 ; CN1CCN(Cc2ccc(C(=O)Cl)cc2)CC1 ; NCCSSCCO ; CN1CCN(Cc2ccc(C(=O)NCCSSCCO)cc2)CC1]

Thionyl chloride (4.26 ml, 0.0426 mol) was added to a solution of 4-(4-methylpiperazin-1-ylmethyl)benzoic acid (1.66 g, 0.00710 mol) in dichloromethane (20 mL) at 25-30° C., and stirring was continued for 2.0 hrs. The reaction mixture was concentrated and quenched with diisopropyl ether (20 mL). The resulting product was filtered and dried to give 4-(4-methylpiperazin-1-ylmethyl)-benzoyl chloride as a brown solid. 2-(2-Aminoethyldisulfanyl)ethanol (1.8 g, 0.00710 mol) and triethyl amine (5.99 ml, 0.0426 mol) was added dropwise to a stirred solution of 4-(4-methylpiperazin-1-ylmethyl)benzoyl chloride in dichloromethane (40 mL) at 22-30° C. The reaction mixture was stirred for 3 hrs. The reaction mixture was quenched with DM (demineralised) water and the organic layer was washed with DM water, dried over anhydrous sodium sulfate and concentrated under vacuum. The resulting residue was purified by column chromatography (10% methanol in dichloromethane) to give title compound as a light brown liquid.

### EXAMPLE 14

### N-[2-(2-Hydroxyethyldisulfanyl)ethyl]-4-(4-methylpiperazin-1-yl)benzamide

The title compound was prepared in a manner similar to example 12 using 4-(4-methylpiperazin-1-yl)benzoic acid instead of 4-(4-methylpiperazin-1-ylmethyl)benzoic acid.

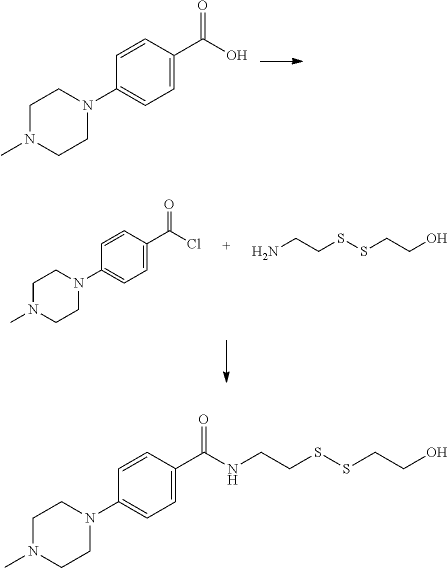 [SMILES: CN1CCN(c2ccc(C(=O)O)cc2)CC1 ; CN1CCN(c2ccc(C(=O)Cl)cc2)CC1 ; NCCSSCCO ; CN1CCN(c2ccc(C(=O)NCCSSCCO)cc2)CC1]

### EXAMPLE 15

### 2-(2-{4-[4-Methylpiperazin-1-yl]benzoylamino}ethyldisulfanyl) ethyl [(4S)-9-tert-butyloxycarbonyloxy-4,11-diethyl-3,4,12,14-tetrahydro-1H-pyrano[3′,4′:6,7] indolizino[1,2-b]quinoline-3,14-(4H,12H)dione-4-yl] carbonate

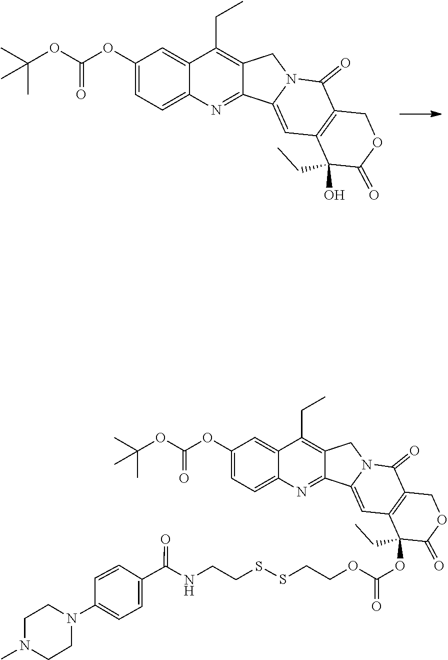 [SMILES: CCc1c2c(nc3ccc(OC(=O)OC(C)(C)C)cc13)-c1cc3c(c(=O)n1C2)COC(=O)[C@]3(O)CC ; CCc1c2c(nc3ccc(OC(=O)OC(C)(C)C)cc13)-c1cc3c(c(=O)n1C2)COC(=O)[C@@]3(CC)OC(=O)OCCSSCCNC(=O)c1ccc(N2CCN(C)CC2)cc1]

Triphosgene (0.246 g, 0.81 mmol) was added to a stirred mixture of 7-ethyl-10-(tert-butyloxycarbonyoxy)camptothecin (1.0 g, 2.03 mmol) and 4-dimethylaminopyridine (0.744 g, 6.09 mmol) in dichloromethane (20 mL) at 20-25° C. The mixture was stirred under a blanket of nitrogen at 20-25° C. After 0.5 hr, N-[2-(2-hydroxyethyldisulfanyl)ethyl]1-4-(4-methylpiperazin-1-yl)benzamide (0.712 g, 2.03 mmol) was added to the reaction mixture, and stirring was continued for 3 hrs. The reaction mixture was quenched with water and dichloromethane layer was separated, washed with water, dried and concentrated in vacuo. The resulting residue was purified by column chromatography on silica gel (5% methanol in dichloromethane) to yield the compound as a light yellow solid.

### EXAMPLE 16

### 2-(2-{4-[4-Methylpiperazin-1-yl]benzoylamino}ethyldisulfanyl) ethyl [(4S)-4,11-diethyl-3,4,12,14-tetrahydro-9-hydroxy-1H-pyrano[3′,4′:6,7]indolizino[1,2-b] quinoline-3,14-(4H,12H)dione-4-yl]carbonate dihydrochloride (Compound I.5)

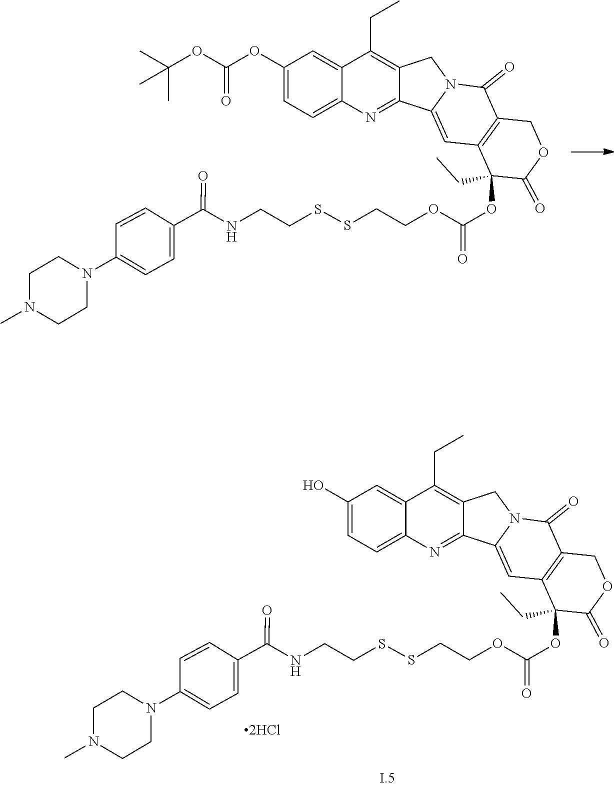 [SMILES: CCc1c2c(nc3ccc(OC(=O)OC(C)(C)C)cc13)-c1cc3c(c(=O)n1C2)COC(=O)[C@@]3(CC)OC(=O)OCCSSCCNC(=O)c1ccc(N2CCN(C)CC2)cc1 ; CCc1c2c(nc3ccc(O)cc13)-c1cc3c(c(=O)n1C2)COC(=O)[C@@]3(CC)OC(=O)OCCSSCCNC(=O)c1ccc(N2CCN(C)CC2)cc1]

Piperidine (0.191 g, 2.229 mmol) was added to a stirred solution of 2-(2-{4-[4-methylpiperazin-1-yl]benzoylamino}ethyldisulfanyl)ethyl [(4S)-9-tert-butyloxy carbonyloxy-4,11-diethyl-3,4,12,14-tetrahydro-1H-pyrano[3′,4′:6,7]indolizino [1,2-b]quinoline-3,14-(4H, 12H)dione-4-yl] carbonate (0.65 g, 0.743 mmol) in 10 ml of acetone at 20-25° C. and stirring was continued for 3 hrs. The reaction mixture was concentrated and residue was stirred with diisopropyl ether. The resulting solid was filtered, washed with diisopropyl ether and purified by column chromatography on silica gel (10 to 15% methanol in dichloromethane). The pure solid was dissolved in a mixture of dichloromethane-methanol and treated with 2 molar equivalent of hydrochloric acid at 10-15° C. The solution was concentrated and the residue was stirred with acetone. The resulting solid was filtered, washed with acetone and dried to yield the title compound as a yellow solid.

¹H-NMR (500 MHz, DMSO-d6, δ ppm): 0.96(t, J=7.39 Hz, 3H), 1.34(t, J=7.59 Hz, 3H), 2.19-2.24(m, 2H), 2.86(d, J=4.70 Hz, 3H), 2.93(t, J=6.77 Hz, 2H), 3.07(t, J=6.50 Hz, 2H), 3.12-3.24(m, 6H), 3.52(d, J=8.00 Hz, 4H), 4.02(d, J-12.29 Hz, 2H), 4.37(t, J=6.11 Hz, 2H), 5.35(s, 2H), 5.57(s, 2H), 7.05(s, 1H), 7.06(d, J=9.00 Hz, 2H), 7.46-7.48(m, 2H), 7.79(d, J=8.87 Hz, 2H), 8.09(d, J=9.80 Hz, 1H), 8.46(m, 1H), 10.92(bs, 1H). Mass (ES+, m/z): 774.32

### EXAMPLE 17

### 2-(2-{4-[4-Methylpiperazin-1-ylmethyl]benzoylamino}ethyldisulfanyl)ethyl [4S)-9-tert-butyloxycarbonyloxy-4,11-diethyl-3,4,12,14-tetrahydro-1H-pyrano[3′,4′:6,7] indolizino[1,2-b]quinoline-3,14-(4H,12H) dione-4-yl] carbonate

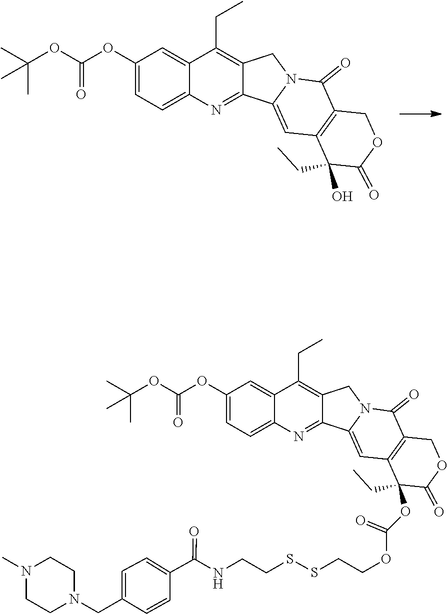 [SMILES: CCc1c2c(nc3ccc(OC(=O)OC(C)(C)C)cc13)-c1cc3c(c(=O)n1C2)COC(=O)[C@]3(O)CC ; CCc1c2c(nc3ccc(OC(=O)OC(C)(C)C)cc13)-c1cc3c(c(=O)n1C2)COC(=O)[C@@]3(CC)OC(=O)OCCSSCCNC(=O)c1ccc(CN2CCN(C)CC2)cc1]

Triphosgene (0.241 g, 0.81 mmol) was added to a stirred mixture of 7-ethyl-10-(tert-butyloxycarbonyoxy)camptothecin (1.0 g, 2.03 mmol) and 4-dimethylaminopyridine (0.744 g, 6.09 mmol) in dichloromethane (50 mL) at 20-25° C. The mixture was stirred under a blanket of nitrogen at 20-25° C. After 0.5 hr, N[2-(2-hydroxyethyldisulfanyl)ethyl]-4-(4-methylpiperazin-1-ylmethyl) benzamide (0.600 g, 1.62 mmol) was added to the reaction mixture, and stirring was continued for 3 hrs. The reaction mixture was quenched with water and the dichloromethane layer was separated, washed with water, dried and concentrated in vacuo. The resulting residue was purified by column chromatography on silica gel (5% methanol in dichloromethane) to yield the compound as a light yellow solid.

### EXAMPLE 18

### 2-(2-{4-[4-Methylpiperazin-1-ylmethyl]benzoylamino}ethyl disulfanyl) ethyl[(4S)-4,11-diethyl-3,4,12,14-tetrahydro-9-hydroxy-1H-pyrano[3′,4′:6,7]indolizino [1,2-b]quinoline-3,14-(4H,12H)dione-4-yl] carbonate dihydrochloride (Compound I.6)

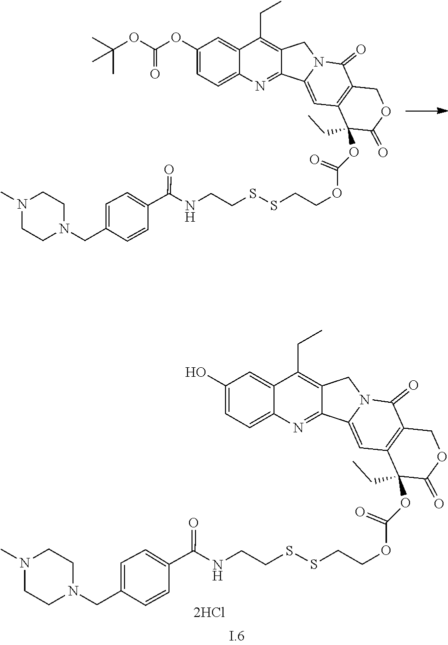 [SMILES: CCc1c2c(nc3ccc(OC(=O)OC(C)(C)C)cc13)-c1cc3c(c(=O)n1C2)COC(=O)[C@@]3(CC)OC(=O)OCCSSCCNC(=O)c1ccc(CN2CCN(C)CC2)cc1 ; CCc1c2c(nc3ccc(O)cc13)-c1cc3c(c(=O)n1C2)COC(=O)[C@@]3(CC)OC(=O)OCCSSCCNC(=O)c1ccc(CN2CCN(C)CC2)cc1]

Piperidine (0.086 g, 1.01 mmol) was added to a stirred solution of 2-(2-{4-[4-butyloxycarbonyloxy-4,11-diethyl-3,4,12,14-tetrahydro-1H-pyrano[3′,4′:6,7]indolizino[1,2-b]quinoline-3,14-(4H,12H)dione-4-yl] carbonate (0.45 g, 0.51 mmol) in 10 ml of acetone at 20-25° C. and stirring was continued for 3 hrs. The reaction mixture was concentrated and residue was stirred with diisopropyl ether. The resulting solid was filtered, washed with diisopropyl ether and purified by column chromatography on silica gel (10 to 15% methanol in dichloromethane). The pure solid was dissolved in mixture of dichloromethane-methanol and treated with 2 molar equivalent of hydrochloric acid at 10-15° C. The solution was concentrated and the residue was stirred with mixture of acetone and diisopropyl ether. The resulting solid was filtered, washed with diisopropyl ether and dried to yield the title compound as a yellow solid.

¹H-NMR (500 MHz, DMSO-d6, δ ppm): 0.96(t, J=7.38 Hz, 3H), 1.34(t, J=7.92 Hz, 3H), 2.17-2.26(m, 2H), 2.86(bs, 3H), 2.93(t, J=6.69 Hz, 2H), 3.08(t, J=6.15 Hz, 2H), 3.10-3.15(m, 2H), 3.43-3.68(m, 10H), 4.38(d, J=5.98 Hz, 2H), 4.49(bs, 2H), 5.34(s, 2H), 5.57(s, 2H), 7.07(s, 1H), 7.48-7.49(m, 2H), 7.80(d, J=8.11 Hz, 2H), 7.93(d, J=8.22 Hz, 2H), 8.09(d, J=9.83 Hz, 1H), 8.78(t, J=6.50 Hz, 1H), 11.94(bs, 1H). Mass (ES+, m/z): 788.32.

### EXAMPLE 19

### [2-(2-{3-Methyl-3-[4-(4-methylpiperazin-1-yl)phenyl]ureido}-ethyl disulfanyl)ethyl]carbamic acid tert-butyl ester

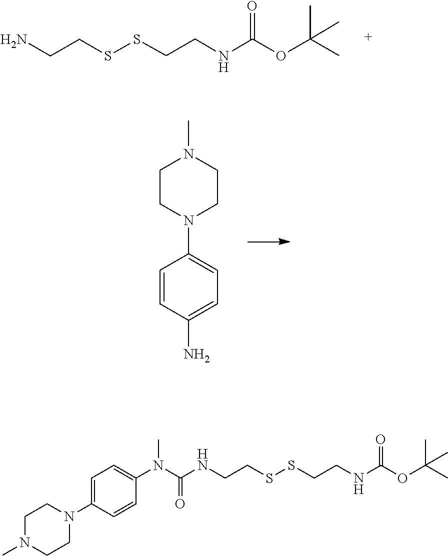

4-Nitrophenylchloroformate (2.97 g, 14.7 mmol) was added to a solution of [2-(2-aminoethyldisulfanyl)ethyl]carbamic acid tert-butyl ester (3.4 g, 13.4 mmol) and triethylamine (2.82 ml, 20.1 mmol) in dichloromethane (50 mL) at 25-30° C., and stirring was continued for 1.0 hr. 4-(4-methylpiperazin-1-yl)aniline (2.56 g, 13.4 mmol) and 4-dimethylaminopyridine (0.1 g, 0.8 mmol) was added to the reaction mixture and stirring was continued for 4.0 hrs. The reaction mixture was quenched with DM water. The dichloromethane layer was separated, dried over anhydrous sodium sulfate and concentrated under vacuum. The crude product was purified by column chromatography on silica gel (5% methanol in dichloromethane) to give title compound as light brown solid.

### EXAMPLE 20

### 3-[2-(2-Aminoethyldisulfanyl) ethyl]-1-methyl-1-[4-(4-methyl piperazin-1-yl) phenyl] urea

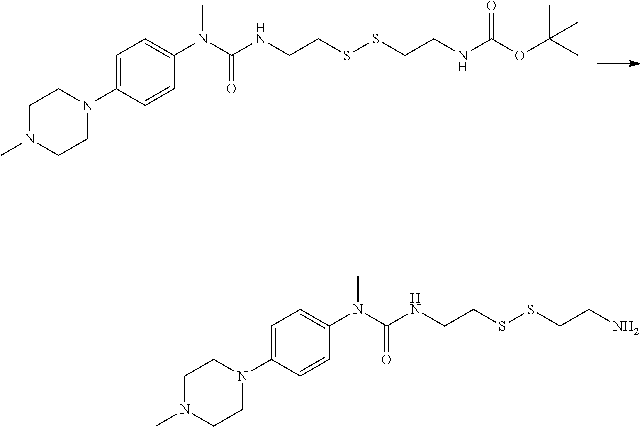

To a solution of [2,-(2-{3-methyl-3-[4-(4-methylpiperazin-1-yl)phenyl]ureido}ethyl disulfanyl)ethyl] carbamic acid tert-butyl ester (1.72 g) in dichloromethane (30 mL) was added trifluoroacetic acid (9 mL) at 25-30° C., and stirring was continued for 3 hrs. The reaction mixture was concentrated and quenched with saturated sodium bicarbonate solution. The product was extracted with dichloromethane. The dichloromethane layer was dried over anhydrous sodium sulfate and concentrated under vacuum. The resulting residue was purified by column chromatography on silica gel (15% methanol in dichloromethane) to give title compound as a light brown solid.

### EXAMPLE 21

### [2-(2-{3-[4-(4-Methylpiperazin-1-yl)phenyl]ureido}ethyldisulfanyl)ethyl] [(4S)-9-tert-butyloxycarbonyloxy-4,11-diethyl-3,4,12,14-tetrahydro-1H-pyrano[3′,4′:6,7] indolizino[1,2-b]quinoline-3,14-(4H,12H) dione-4-yl] carbamate

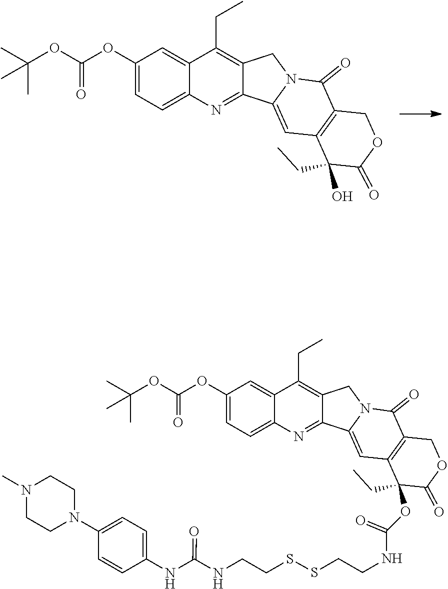 [SMILES: CCc1c2c(nc3ccc(OC(=O)OC(C)(C)C)cc13)-c1cc3c(c(=O)n1C2)COC(=O)[C@]3(O)CC ; CCc1c2c(nc3ccc(OC(=O)OC(C)(C)C)cc13)-c1cc3c(c(=O)n1C2)COC(=O)[C@@]3(CC)OC(=O)NCCSSCCNC(=O)Nc1ccc(N2CCN(C)CC2)cc1]

Triphosgene (0.48 g, 1.62 mmol) was added to a stirred mixture of 7-ethyl-10-(tert-butyloxycarbonyoxy)camptothecin (2 g, 4.06 mmol) and 4-dimethylaminopyridine (1.48 g, 12.2 mmol) in dichloromethane (50 mL) at 15-20° C. The mixture was stirred under a blanket of nitrogen at 15-20° C. After 0.5 hr, 1-[2-(2-aminoethyldisulfanyl)ethyl]-3-[4-(4-methylpiperazin-1-yl)phenyl]urea (1.34 g, 3.65 mmol) was added to the reaction mixture, and stirring was continued for 3 hrs. The reaction mixture was quenched with water and dichloromethane layer was separated, washed with water, dried and concentrated in vacuo. The resulting residue was purified by column chromatography on silica gel (10% methanol in dichloromethane) to yield the compound as a light yellow solid.

### EXAMPLE 22

### [2-(2-{3-[4-(4-Methylpiperazin-1-yl)phenyl]ureido}-ethyldisulfanyl)ethyl] [(4S)-4,11-diethyl-3,4,12,14-tetrahydro-9-hydroxy-1H-pyrano[3′,4′: 6,7]indolizino[1,2-b]quinoline-3,14-(4H,12H)dione-4-yl] carbamate hydrochloride. (Compound I.7)

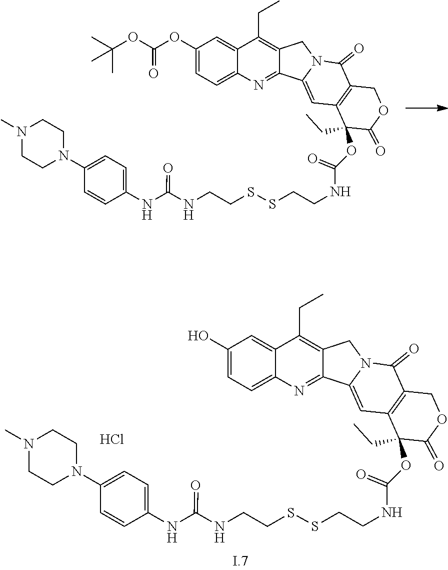 [SMILES: CCc1c2c(nc3ccc(OC(=O)OC(C)(C)C)cc13)-c1cc3c(c(=O)n1C2)COC(=O)[C@@]3(CC)OC(=O)NCCSSCCNC(=O)Nc1ccc(N2CCN(C)CC2)cc1 ; CCc1c2c(nc3ccc(O)cc13)-c1cc3c(c(=O)n1C2)COC(=O)[C@@]3(CC)OC(=O)NCCSSCCNC(=O)Nc1ccc(N2CCN(C)CC2)cc1 ; Cl]

Piperidine (0.125 g, 1.46 mmol) was added to a stirred solution of [2-(2-{3-[4-(4-methylpiperazin-1-yl)phenyl]ureido}ethyldisulfanyl)ethyl] [(4S)-9tert-butyloxycarbonyloxy-4,11-diethyl-3,4,12,14-tetrahydro-1H-pyrano[3′,4′:6,7]indolizino[1,2-b]quinoline-3,14-(4H,12H)dione-4-yl] carbamate (0.65 g, 0.73 mmol) in 10 ml of acetone at 20-25° C. and stirring was continued for 4 hrs. The reaction mixture was quenched with diisopropyl ether. The resulting solid was filtered, washed with diisopropyl ether and purified by column chromatography on silica gel (10 to 20% methanol in dichloromethane). The pure solid was dissolved in a mixture of dichloromethane-methanol and treated with 1 molar equivalent of hydrochloric acid at 10-15° C. The solution was concentrated and the residue was stirred with acetone. The resulting solid was filtered, washed with acetone and dried to yield the title compound as a yellow solid.

¹H-NMR (500 MHz, DMSO-d6, δ ppm): 1.00(t, J=7.19 Hz, 3H), 1.35(t, J=7.37 Hz, 3H), 2.40-2.41(m, 1H), 2.78(m, 1H), 2.85-2.86(m, 5H), 3.00-3.03(m, 4H), 3.13-3.20(m, 4H), 3.31-3.38(m, 3H), 3.51(d, J-11.69 Hz, 2H), 3.70(d, J-12.80 Hz, 2H), 3.87-3.89(m, 2H), 4.83(d, J-11.67 Hz, 1H), 4.90(d, J-11.67 Hz, 1H), 5.32(s, 2H), 6.37(bs, 1H), 6.93(d, J=8.88 Hz, 2H), 7.31(d, J=8.80 Hz, 2H),7.35(S, 1H), 7.47-7.49(m, 2H), 8.12(d, J=9.52 Hz, 1H), 8.55(s, 1H), 10.63(bs, 1H). Mass (ES+, m/z): 788.23

### Buffer Stability

The representative compounds were first dissolved in a minimum quantity of DMSO in a volumetric flask and diluted up to the mark with diluent (water: acetonitrile 30:70). These solutions were further diluted to 10-fold volume with phosphate buffer at three different pH (4.7, 6 and 7.4) externally to achieve the concentration of 200 μg/ml and stability was checked at different time points by keeping the samples in an incubator at 37° C. After the elapsed time, the buffer samples were diluted 4 times with acetonitrile and injected into an HPLC system. Quantification of representative compounds was done by HPLC.

HPLC method: Chromatographic separation was achieved on a Hypersil BDS C18 (100×4.6 mm, 5 μ) column using 25 mM KH₂PO₄ buffer (pH 7 with TEA): acetonitrile, 90:10 v/v as mobile phase-A and 25 mM KH₂PO₄ buffer (pH 7 with TEA): acetonitrile 30:70 v/v as mobile phase-B. The gradient started with initially 5% B to 45% B in 10 mins followed by 80% B in 22 mins and held up to 25 mins and then returned to initial conditions at 26 mins and continued up to 30 mins. The injection volume was kept 10 μL and the flow rate was kept at 1.0 mL/min. The UV-Vis Detector was set at 220 nm, the column oven was set at 37° C. and the sample cooler was set at 37° C. Total chromatographic runtime was 30 min. 0 Hr standard area was considered as 100% for calculation.

The % unconverted test compound in buffer samples at different time points is shown in below Table 2.

TABLE 2
Buffer Stability of Compounds of Formula I

| | | % of Unconverted Compounds of Formula I | | | | |
|---|---|---|---|---|---|---|
| Compound | pH | 0 hr | 2 hr | 4 hr | 6 hr | 8 hr |
| I.1 | 4.7 | 100 | 96.3 | 97.4 | 100.0 | 100 |
| | 6.0 | 100 | 95.4 | 97.1 | 97.2 | 97.1 |
| | 7.4 | 100 | 97.7 | 95.5 | 100.0 | 100 |
| I.2 | 4.7 | 100 | 97.3 | 100 | 100 | 100 |
| | 6.0 | 100 | 95.5 | 98.5 | 100 | 100 |
| | 7.4 | 100 | 90.1 | 93.5 | 88.7 | 91.6 |
| I.3 | 4.7 | 100 | 100 | 100 | 100 | 100 |
| | 6.0 | 100 | 93.8 | 97.8 | 98.2 | 100 |
| | 7.4 | 100 | 100 | 100 | 100 | 100 |
| I.5* | 4.7 | 100 | 82.5 | 86.3 | 79.9 | 77.2 |
| | 6.0 | 100 | 94.1 | 100 | 100 | 98.4 |
| | 7.4 | 100 | 88.0 | 82.8 | 82.5 | 82.4 |
| I.6* | 4.7 | 100 | 96.8 | 89.8 | — | 89.0 |
| | 6.0 | 100 | 89.9 | 89.4 | 82.5 | 84.3 |
| | 7.4 | 100 | 90.7 | 81.4 | 83.7 | 84.6 |

*Diluted with phosphate buffer to achieve 100 μg/ml conc. and after elapsed time buffer samples diluted 4 times with methanol: water (70:30) and analysed by HPLC method.

The stability of the compounds of Formula I at different pH is demonstrated by the above results. Even after 8 h of incubation in buffer having different pH, most of the test compounds showed only as much as about 10% degradation. A similar trend is observed under acidic pH. Thus, the compounds are expected to be stable under conditions of the human gastro-intestinal tract and thus can be suitable for oral administration.

### In-Vitro Cancer Cell Growth Inhibition Assay

The compounds of the present invention were evaluated for their ability to inhibit the growth of various cell line models of small cell lung cancer (SCLC), colon cancer, pancreatic cancer and triple negative breast cancer (TNBC) in-vitro. Details of the cell lines and their respective complete growth media are described in Table 3. The growth inhibition assay was carried out as described below. Briefly, cells in complete growth media were seeded in 96-well plates at appropriate cell densities (seeding density details described in Table 3) and incubated at 37° C., 5% CO₂ (3-4 hours for NCI-H526, NCI-H69, NCI-H187 and overnight for the rest of the cells). Serial dilutions of test compound in DMSO were added to the cells while maintaining the final DMSO concentration of 0.4%-0.5% in the well. Plates were incubated for 96-144 h (the exact duration for each assay is described in Table 3) at 37° C., 5% CO₂. Subsequently, MTT (final concentration 0.5 mg/mL in media) was incubated with the cells at 37° C., 5% CO₂ for 4-5 hours. The formazan crystals were dissolved overnight at 37° C., 5% CO₂ using 100 μL extractant (10% SDS in 0.01N HCl) and quantified using absorbance at 570 nm with reference wavelength 630 nm. Growth inhibition was represented as percent decrease in absorbance compared to vehicle treated cells. The results of the growth inhibition are shown in Table 4.

TABLE 3
Conditions of In-Vitro Cancer Cell Growth Inhibition Assay

| | | Seeding Density | Assay | |
|---|---|---|---|---|
| Cell Line | Growth Medium | (cells/well) | Duration | |
| NCI-H526 | RPMI-1640 medium; 10% FBS | 25,000 | 72 | hr |
| NCI-H69 | RPMI-1640 medium; 10% FBS | 40,000 | 144 | hr |
| NCI-H187 | RPMI-1640 medium; 10% FBS | 50,000 | 168 | hr |
| HT-29 | McCoy's 5A medium; 10% FBS | 10000 | 96 | hr |
| PANC-1 | DMEM medium; 10% FBS | 8000 | 96 | hr |
| MX-1 | DMEM:F12 medium; 10% FBS | 7000 | 144 | hr |
| MDA-MB-231 | DMEM medium; 10% FBS | 5000 | 96 | hr |
| MDA-MB-468 | DMEM medium; 10% FBS | 5000 | 96 | hr |

TABLE 4
In-Vitro Cancer Cell Growth Inhibition on Various Cell Lines

| | | NCI | NCI | NCI | | MDA- | | MDA- |
|---|---|---|---|---|---|---|---|---|
| | HT-29 | H69 | H187 | H526 | PANC-1 | MB-231 | MX-1 | MB-468 |
| # | IC₅₀ (nM) | | | | | | | |
| Irinotecan | 13956 | 473 | 385 | 750 | 22997 | 21705.7 | 5911.5 | 4580.5 |
| HCl trihydrate | | | | | | | | |
| Cmpd I.2 | 31.0 | 1.3 | 0.27 | 1.58 | 508 | 111.65 | 5.9 | 6.6 |
| Cmpd I.3 | 43.7 | 1.2 | 0.68 | 1.91 | 862 | — | — | — |
| Cmpd I.4 | — | — | — | — | — | 129.15 | 6.9 | 8.2 |
| Cmpd I.5 | 48.3 | 1.3 | 0.77 | 2.06 | 910 | 118.65 | 4.6 | 9.3 |
| Cmpd I.6 | 15.7 | 2.3 | 0.23 | — | 1142 | 157.5 | 5.4 | 13.7 |

As can be seen from the Table 4, Compounds I.2, I.3, I.4, I.5 and I.6 showed better in-vitro antitumor activities in HT-29 cells, NCI H69 cells, NCI H187 cells, NCI H526 cells, PANC-1 cells, MDA-MB-231 cells, MX-1 cells and MDA-MB468 cells than irinotecan.

### In-Vitro Stability in Mice Tumor Lysate

Compound I.4, irinotecan hydrochloride and SN-38 were spiked individually into mice tumor homogenate (small cell lung cancer cell line NCI-H1048 tumor homogenate in 20% phosphate buffer pH 5.5) externally to achieve the concentration of 2000 ng/mL and stability was checked at different time points by keeping stability samples in an incubator at 37° C. Aliquot of 100 μL were taken from stability samples in pre-labelled micro-centrifuge tubes. 5 μL of cetirizine working internal standard (5 μg/mL) was added to each tube and vortexed well. 1 mL acetonitrile was added to each tube and vortexed well and then centrifuge at 10000 RPM for 5 min at room temperature. The supernatant was collected in ria vials and evaporated to dryness under nitrogen stream. The samples were reconstituted in 1 mL of 0.1% formic acid in water: acetonitrile 30:70 v/v. The prepared samples were analyzed using LC-MS/MS method. Quantification was done against SN-38 standard, prepared at 2000 ng/mL concentration. SN-38 standard prepared by spiking 5 μL of SN-38 working standard to 95 μL of blank mice tumor homogenate to achieve 2000 ng/mL concentration and vortexed well and processed as described.

LC-MS/MS Method: Chromatographic separation was achieved on Inertsil C8-3 (50×4.6 mm, 5 μ) with a flow rate of 250 μL/min and an injection volume of 10 μL. The sample cooler was maintained at 10° C. The column oven temperature was set to 40° C. The mobile phase consisted of 0.1% formic acid in Milli Q water and acetonitrile in the ratio of 30:70 v/v, respectively. The retention time of Compound I.4, Irinotecan, SN-38 and internal standard was about 1.32, 1.73, 2.37 and 1.72 min, respectively. The overall chromatographic run time was 4.0 minutes.

Detection was performed by tandem mass spectrometry (TSQ Quantum, Discovery MAX, Thermo Electron Corporation) and peak areas were integrated using LCquan software version 2.9 QF1. The detector was set on SRM mode where transition of 804.170 m/z→263.020 m/z (CE 37), 331.030 m/z (CE 44), 347.070 m/z (CE 43) was monitored for Compound I.3, 587.300 m/z→124.050 m/z (CE 33) was monitored for irinotecan, 393.300 m/z→212.360 m/z (CE 35), 306.360 m/z (CE 31), 348.980 m/z (CE 24) for SN-38 and 389.160 m/z→200.923 m/z (CE 20) was monitored for the internal standard.

The formation of SN-38 and percentage remaining of compound I.4, irinotecan and SN-38 in tumor homogenate samples at different time points is shown in below table 5. 0 hr standard area was considered as 100% for calculation.

TABLE 5
Stability in Tumor Lysate

| | | Concentration | | Concentration | |
|---|---|---|---|---|---|
| | | of SN-38 | | of SN-38 | |
| | % Remaining | formed from | | formed from | % Remaining |
| | of Compound | compound I.4 | % Remaining | Irinotecan | of SN-38 in |
| Time | I.4 | (ng/mL) | of Irinotecan | (ng/mL) | tumor lysate |
| 0 hr | 100.0 | 370.2 | 100.0 | 0.0 | 100.0 |
| 1 hr | 0.0 | 585.5 | 110.5 | 9.4 | 94.9 |
| 2 hr | 0.0 | 581.8 | 95.4 | 17.5 | 90.7 |
| 4 hr | 0.0 | 594.1 | 93.1 | 22.8 | 100.1 |
| 6 hr | 0.0 | 615.0 | 82.3 | 25.5 | 88.9 |
| 8 hr | 0.0 | 504.3 | 75.6 | 29.6 | 91.4 |

From the results, it is clear that, the compound I.4 rapidly cleaved to give active SN-38 within 1 hour after incubation, whereas more than 75% of irinotecan remained in tumor lysate even after 8 hours of the incubation.

In summary, these studies show that the compounds of the present invention not only have good water solubility and stability, but also have considerable in vitro cytotoxicity and are more potent than irinotecan. The compounds described herein can rapidly be cleaved in tumor microenvironments to deliver SN-38 so that it can significantly inhibit cancer cell proliferation.

The compounds of the present invention are stable in buffer solution at pH 4.7, pH 6.0 and pH 7.4 simulating the stability under the condition of human gastro-intestinal tract and thus can be suitable for oral administration. The compounds of the present invention can be formulated in oral dosage forms.

All references cited herein are hereby incorporated by reference.

1. A compound of Formula I 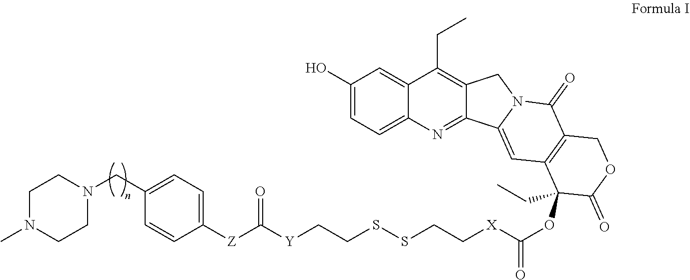
or a pharmaceutically acceptable salt thereof, wherein
X is —NH—, —O— or —CH₂—;
Y is —NH—, —O— or —CH₂—;
Z is absent, —NH— or —N(C₁₋₃ alkyl)-; and
n is an integer selected from 0 or 1.

2. The compound of claim 1, wherein
X is —O—; and
Y is —NH— or —O—.

3. The compound of claim 1, wherein
X is —O—;
Y is —O—;
Z is —NH— or —N(C₁₋₃ alkyl)-; and
n is 0.

4. The compound of claim 1, wherein
X is —O—;
Y is —O—;
Z is —N(C₁₋₃alkyl)-; and
n is 0.

5. The compound of claim 4, wherein Z is —N(CH₃)—.

6. A compound selected from
4-[3-(4-(4-Methylpiperazin-1-yl)phenylcarbamoyl)propyldisulfanyl][(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]butyrate;
2-(2-{N-[4-(4-Methylpiperazin-1-yl)phenyl]carbamoyloxy}ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]carbonate;
2-(2-{N-Methyl-N-[4-(4-methylpiperazin-1-yl)phenyl]carbamoyloxy} ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl]carbonate;
2-(2-{4-[4-Methylpiperazin1-yl]benzoylamino}ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate;
2-(2-{4-[4-Methylpiperazin-1-ylmethyl]benzoylamino}ethyldisulfanyl)ethyl [(4S)-4,11-diethyl-3,4,12,14-tetrahydro-9-hydroxy-1H-pyrano[3′,4′:6,7]indolizino[1,2-b]quinoline-3,14-(4H,12H)dione-4-yl] carbonate;
[2-(2-{3-[4-(4-Methylpiperazin-1-yl)phenyl]ureido}ethyldisulfanyl)ethyl] [(4S)-4,11-diethyl-3,4,12,14-tetrahydro-9-hydroxy-1H-pyrano[3′,4′:6,7]indolizino[1,2-b]quinoline-3,14-(4H ,12H)dione-4-yl] carbamate;
and pharmaceutically acceptable salts thereof.

7. A pharmaceutical composition comprising the compound of claim 1 and a pharmaceutically acceptable carrier, diluent, or excipient.

8. A method of treatment of a cancer selected from the group consisting of lung cancer, breast cancer, colon cancer, rectal cancer, prostate cancer, melanoma, pancreatic cancer, stomach cancer, liver cancer, brain cancer, kidney cancer, cancer of the uterus, cancer of the cervix, ovarian cancer, cancer of the urinary tract, gastrointestinal cancer, urothelial cancer, head and neck cancer, thyroid cancer, esophageal cancer, endometrial cancer, and cholangiocarcinoma, comprising administering to a subject in need thereof an effective amount of the compound of claim 1.

9. The method of treatment of claim 8, wherein the cancer is selected from non-small cell lung cancer, triple negative breast cancer, ovarian cancer, colon cancer and cholangiocarcinoma.

10. (canceled)

11. (canceled)

12. A method of manufacturing a medicament for treating a cancer selected from a group consisting of lung cancer, breast cancer, colon cancer, rectal cancer, prostate cancer, melanoma, pancreatic cancer, stomach cancer, liver cancer, brain cancer, kidney cancer, cancer of the uterus, cancer of the cervix, ovarian cancer, cancer of the urinary tract, gastrointestinal cancer, urothelial cancer, head and neck cancer, thyroid cancer, esophageal cancer, endometrial cancer, and cholangiocarcinoma, comprising combining the compound of claim 1 and a pharmaceutically acceptable carrier, diluent, or excipient.

13. The method of claim 13, wherein the cancer is selected from non-small cell lung cancer, triple negative breast cancer, ovarian cancer, colon cancer and cholangiocarcinoma.
